(19) United States
(12) Patent Application Publication
(10) Pub. No.: US 2006/0116689 A1
(43) Pub. Date: Jun. 1, 2006

(54) Surgical instrumentation and method for treatment of a spinal structure

(75) Inventors: Willliam J. Albans, Cordova, TN (US); Randy N. Allard, Germantown, TN (US); Jill M. Embry, Somerville, TN (US); Jon C. Serbousek, Memphis, TN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc., Wilmington, DE (US)

(21) Appl. No.: 11/154,319

(22) Filed: Jun. 16, 2005

Related U.S. Application Data
(60) Provisional application No. 60/580,055, filed on Jun. 16, 2004.

(51) Int. Cl. A61F 2/34

(52) U.S. Cl. 606/92

(57) ABSTRACT

Embodiments of the invention include instrumentation and methods for treatment of a spinal structure or other orthopedic structures. An elongate member including a deformable distal portion having an initial configuration for placement within a spinal structure or other orthopedic structures, and a deformed configuration wherein the distal portion is outwardly deformed is provided. The elongated member may be used to access the interior of the spinal structure or other orthopedic structures and to manipulate tissue within the structure.

### CROSS REFERENCE TO RELATED APPLICATIONS

The present application claims the benefit of U.S. Provisional Patent Application Ser. No. 60/580,055 filed on Jun. 16, 2004, the contents of which are hereby incorporated by reference in their entirety.

### TECHNICAL FIELD

The present invention relates generally to the field of surgical instrumentation and methods, and more particularly relates to instrumentation and methods for the repair of vertebral bodies and other orthopedic structures.

### BACKGROUND

Various instruments and methods for the treatment of certain compression-type bone fractures and other osteoporotic and/or non-osteoporotic conditions have been developed. Such methods generally include a series of steps performed by a surgeon to correct and stabilize the compression fracture. In some cases, an access opening is formed in the bone to be treated followed by the insertion of an inflatable balloon-like device through the access opening and into an interior portion of the bone. Inflation of the balloon-like device may result in compaction of the bone marrow against the inner cortical wall of the bone, thereby resulting in the formation of a cavity in the bone and reduction of the compression fracture. The balloon-like device may then be deflated and removed from the bone. A biocompatible filling material, such as methylmethacrylate cement or a synthetic bone substitute, is sometimes delivered into the bone cavity and allowed to set to a hardened condition to provide internal structural support to the bone.

### SUMMARY

An embodiment of the invention is a kit for treatment of the spine. The kit may include at least one cannula for maintaining a passageway to a portion of the spine to be treated and a surgical instrument for providing surgical access to the spine, the instrument being operable through the cannula. The kit of some embodiments also has a bone filler injector and a tube that provides a conduit between the bone filler injector and the cannula. The tube is extendable through the cannula to a position adjacent to the portion of the spine to be treated in some embodiments.

Yet another embodiment of the invention is a method of performing a biopsy with a medical instrument comprising a cannula member extending along a longitudinal axis and including a distal portion, with the cannula member defining an axial passage and a transverse opening positioned adjacent the distal portion and communicating with the axial passage, and an actuator member removably positioned within the axial passage of the cannula member and including a deformable portion positioned adjacent the transverse opening, and with the deformable portion being transitionable between an initial configuration for placement within a spinal structure and a deformed configuration defining a transverse projection extending through the transverse opening in the cannula member. Embodiments of the method also include selectively removing tissue on which a biopsy is to be accomplished from the cannula member.

Still another embodiment of the invention is a method for treatment of the spine. The method includes at least the acts of providing an instrument defining a cannula passage extending along a longitudinal axis and including a deformable distal portion having an insertion configuration and a deformed configuration, positioning the distal portion of the instrument within a spinal structure while in the insertion configuration, transitioning the distal portion of the instrument toward the deformed configuration while simultaneously rotating the instrument about the longitudinal axis to form a volume of loosened tissue within the spinal structure, and delivering a material through the cannula passage and into the spinal structure.

Another embodiment of the invention is a method for treatment of the spine, comprising providing an instrument defining a cannula passage extending along a longitudinal axis and including a deformable distal portion having an insertion configuration and a deformed configuration, and positioning the distal portion of the instrument within a spinal structure while in the insertion configuration. The instrument is activated to loosen tissue within the spinal structure. The method also includes removing a portion of the loosened tissue from the spinal structure, and delivering a material through the cannula passage and into the spinal structure.

Yet another embodiment of the invention is a method for treatment of the spine. The method includes at least the acts of providing an instrument defining a cannula passage extending along a longitudinal axis, positioning the distal portion of the instrument within a spinal structure while in the insertion configuration, and delivering a first portion of filler material through a tube extended through the cannula to a distal end of the accessible portion of the spinal structure. The tube is withdrawn proximally relative to the cannula and a second portion of filler material is delivered through the tube and into the spinal structure.

### BRIEF DESCRIPTION OF THE DRAWINGS

FIG. 1 is a perspective view of a surgical instrument according to one form of the present invention.

FIG. 2 is an exploded side view of a distal end portion of the surgical instrument depicted in FIG. 1.

FIG. 3 is an exploded side view of a proximal end portion of the surgical instrument depicted in FIG. 1.

FIG. 4 is a broken cross-sectional side view of the surgical instrument depicted in FIG. 1.

FIG. 5 is a perspective view of the distal end portion of the surgical instrument depicted in FIG. 1, as shown in an initial configuration.

FIG. 6 is a perspective view of the distal end portion depicted in FIG. 5, as shown in a deformed configuration.

FIG. 7 is a perspective view of the distal end portion of a surgical instrument according to another form of the present invention, as shown in an initial configuration.

FIG. 8 is a perspective view of the distal end portion depicted in FIG. 7, as shown in a deformed configuration.

FIG. 9 is a perspective view of the distal end portion of a surgical instrument according to another form of the present invention, as shown in an initial collapsed configuration.

FIG. 10 is a perspective view of the distal end portion depicted in FIG. 9, as shown in a partially expanded configuration.

FIG. 11 is a perspective view of the distal end portion depicted in FIG. 9, as shown in a fully expanded configuration.

FIG. 12 is a partial cross-sectional side view of a spinal column illustrating treatment of a vertebral body using the surgical instrument illustrated in FIG. 1.

FIG. 13 is a perspective view of a surgical instrument according to another form of the present invention.

FIG. 14 is an exploded perspective view of the surgical instrument illustrated in FIG. 13.

FIG. 15 is the surgical instrument illustrated in FIG. 13, as shown in an initial configuration for insertion of the distal portion of the instrument into a vertebral body.

FIG. 16 is the surgical instrument illustrated in FIG. 13, as shown in an expanded configuration for forming a cavity within the vertebral body.

FIG. 17 is the surgical instrument illustrated in FIG. 13, as shown in a delivery configuration for conveying a filling material into the cavity formed within the vertebral body.

FIG. 18 is a perspective view of a surgical instrument according to another form of the present invention.

FIG. 19 is a side view of a surgical instrument according to another form of the present invention.

FIG. 20 is an end view of the proximal end of the surgical instrument illustrated in FIG. 19.

FIG. 21 is a cross-sectional view of the surgical instrument illustrated in FIG. 20, as taken along line 21-21 of FIG. 20.

FIG. 22 is a side view of a surgical instrument according to another form of the present invention.

FIG. 23 is a partial cross-sectional view of the surgical instrument illustrated in FIG. 22, as taken along line 23-23 of FIG. 22.

FIG. 24 is a partially exploded side view of a surgical instrument according to another form of the present invention.

FIG. 25 is a partially exploded side view of a surgical instrument according to another form of the present invention.

FIG. 26 is a side view of a surgical instrument according to another form of the present invention.

FIG. 27 is a partial cross-sectional view of the surgical instrument illustrated in FIG. 26, as taken along line 27-27 of FIG. 26.

### DESCRIPTION

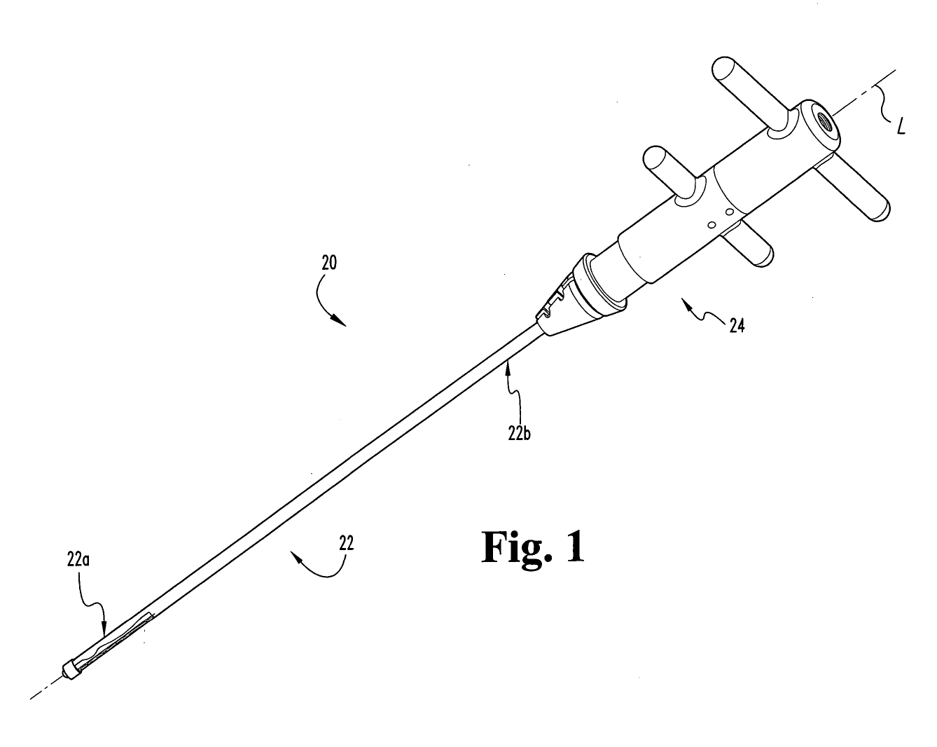

Referring to FIG. 1, shown therein is an instrument 20 for treatment of the spine according to one form of the present invention. Instrument 20 is particularly useful for placement adjacent a spinal structure and selective displacement of at least a portion of the spinal structure. In one embodiment of the invention, the spinal structure is a vertebral body. It should be understood that instrument 20 may be used in intrabody applications such as, for example, a vertebroplasty procedure to compact cancellous bone within the vertebral body and/or to reduce a compression fracture of the vertebral body. Additionally, it should be understood that instrument 20 may be used in interbody applications such as, for example, to distract a space between adjacent vertebral bodies, such as the vertebral disc space. It should further be understood that in other embodiments of the invention, the spinal structure may be comprised of a spinal implant such as, for example, a cage device, or any other structure used in association with treatment of the spine. Additionally, although instrument 20 is illustrated and described in the context of treatment of a human spine, it should be understood that instrument 20 may be used to treat other animals. It should further be understood that instrument 20 may be used in association with applications outside of the spinal field such as, for example, to treat other types of bony structures.

Instrument 20 is generally comprised of an elongate member 22 extending generally along a longitudinal axis L and having a distal end portion 22a and a proximal end portion 22b. Although the illustrated embodiment depicts elongate member 22 as having a generally linear, unitary configuration, it should be understood that elongate member 22 may take on other configurations as well, such as, for example, a curvilinear configuration or a hinged configuration. Instrument 20 also includes an actuator mechanism 24 coupled to the proximal end portion 22b of elongate member 22. As will be discussed in greater detail below, the distal end portion 22a is deformable and is configured to outwardly expand in response to a mechanically induced force. Such force may be effected, for example, by the selective actuation of actuator mechanism 24.

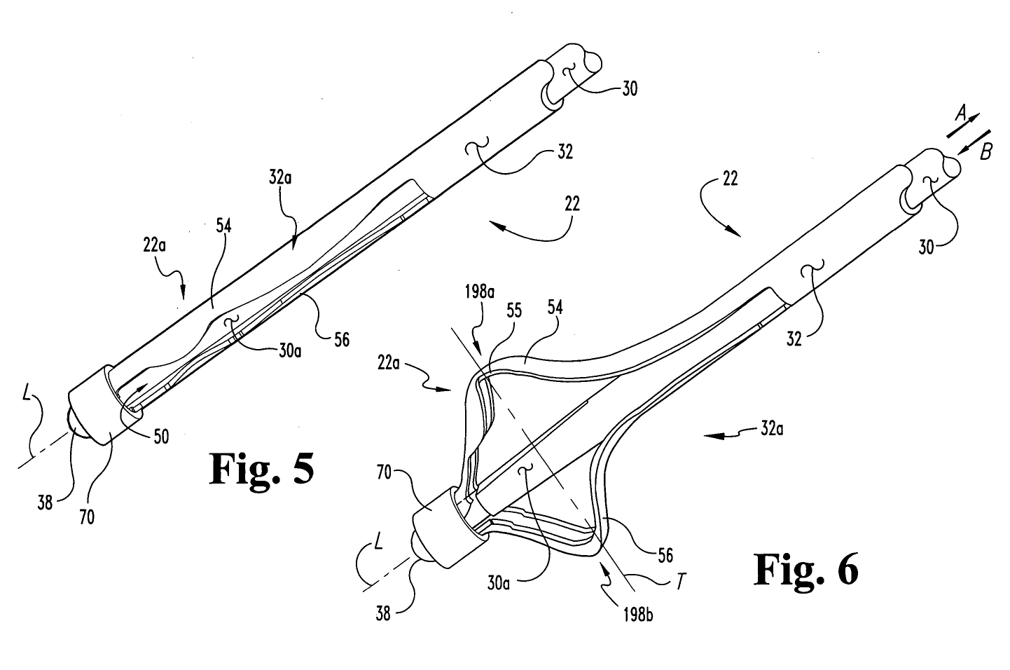

As shown in FIGS. 5 and 6, the distal end portion 22a is reformable between an initial configuration (FIG. 5) and a deformed configuration (FIG. 6). As used herein, the term “initial configuration” is broadly defined to encompass a structural configuration of elongate member 22 that is suitable for placement adjacent a spinal structure, and the term “deformed configuration” is broadly defined to encompass a structural configuration of elongate member 22 that is suitable for preparation or displacement of at least a portion of the spinal structure. As discussed above, in one embodiment of the inventions the spinal structure is a vertebral body, and preparation of the vertebral body could be associated with either intrabody or interbody applications.

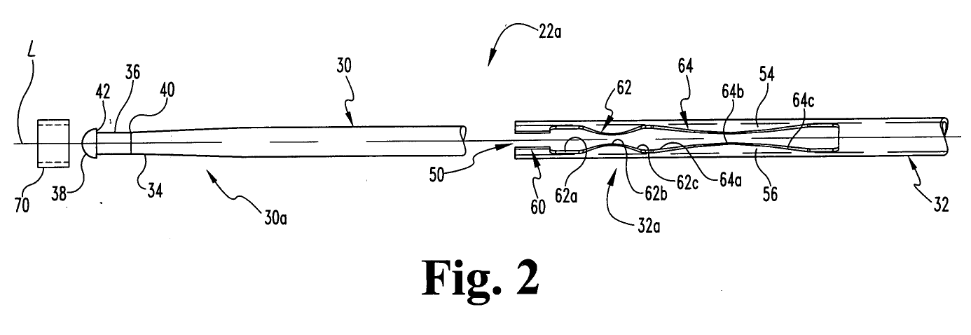

Referring to FIG. 2, shown therein are further details regarding the elongate member 22, and more specifically the deformable distal end portion 22a of elongate member 22. In one embodiment of the invention, the elongate member 22 is comprised of an inner rod member 30 and an outer sleeve member 32. The illustrated embodiment of the inner rod 30 is formed of a substantially rigid medical grade material such as, for example, titanium or stainless steel. The distal end portion 30a of rod 30 includes a tapered portion 34, a reduced cross-section intermediate portion 36, and a rounded distal end portion 38. In one embodiment, the intermediate portion 36 has a diameter somewhat smaller than the diameter of the tapered portion 34 and the rounded distal end portion 38 so as to define a pair of opposing shoulders 40, 42. Although rod 30 has been illustrated and described as having a substantially circular cross section, it should be understood that other shapes and configurations are also contemplated as being within the scope of the invention including, for example, elliptical, square, rectangular or other polygonal configurations.

The outer sleeve 32 as illustrated has a tubular configuration defining an inner passage extending therethrough generally along longitudinal axis L and sized to slidably receive rod 30. Sleeve 32 may be formed of a flexible material that is capable of facilitating deformation from an initial configuration toward a deformed configuration. Additionally, the sleeve 32 illustrated is formed of an elastic material that is capable of facilitating elastic deformation from the initial configuration toward the deformed configuration and reformation back toward the initial configuration. Sleeve 32 may be formed of materials including, but not limited to, titanium, stainless steel, an elastomer, a polymer, a rubber, a composite material or a shape-memory material. Although the entire length of sleeve 32 may be formed of a flexible, elastic material, it should be understood that only the distal end portion 32a of sleeve 32 need be formed of such material, with the remainder of sleeve 32 being formed of any suitable medical grade material. Moreover, although outer sleeve 32 is illustrated as having a substantially tubular configuration, it should be understood that other shapes and configurations of sleeve 32 are also contemplated as being within the scope of the present invention. Additionally, although sleeve 32 has been illustrated and described as being formed as a single-piece, unitary structure, it should be understood that the distal end portion 32a could be formed separately from the remainder of sleeve 32, and coupled together by any known method, such as, for example, by fastening, welding or adhesion.

The distal end portion 32a of sleeve 32 includes at least one slot 50 extending generally along longitudinal axis L, and may include at least a pair of slots 50 and 52 (not shown) disposed generally opposite one another so as to define a pair of longitudinally extending flexible strips of material 54, 56. It should be understood, however, that the distal end portion 32a of sleeve 32 could be configured to define any number of longitudinally extending slots, including three or more slots, which would in turn define a corresponding number of longitudinally extending flexible strips of material. It should further be understood that distal end portion 32a may include a number of slots disposed at various axial locations along longitudinal axis L. As will be described below, the slots 50, 52 are provided to facilitate outward buckling of the distal end portion 32a of sleeve 32 in at least one predetermined direction upon the selective actuation of the actuator mechanism 24.

In the illustrated embodiment, the slots 50, 52 are substantially identical in shape and configuration, and thus only slot 50 will be described in detail. However, it should be understood that slots 50, 52 may take on different shapes and configurations. Slots 50, 52 and strips of material 54, 56 are illustrated as having a predetermined shape to provide a degree of control over the outward buckling of the strips of material 54, 56. In one embodiment of the invention, the slots 50, 52 and strips of material 54, 56 have an irregular shape. Slot 50 includes a relatively narrow and straight slot portion 60, a first hourglass-shaped slot portion 62 formed by a first series of arcuate portions, and a second hourglass-shaped slot portion 64 formed by a second series of arcuate portions. As will become apparent below, the widened areas of the hourglass-shaped portions 62 and 64 serve as bending or flexion points to control the outward deformation of the flexible strips of material 54, 56.

The straight slot portion 60 extends longitudinally from the distal end of sleeve 32. The first hourglass-shaped portion 62 extends longitudinally from slot portion 60 and includes a first widened area 62a, a narrowed area 62b, and a second widened area 62c. The second hourglass-shaped portion 64 extends longitudinally from the first hourglass-shaped portion 62 and includes a first widened area 64a, a narrow area 64b, and a second widened area 64c. Although a specific configuration of slots 50, 52 have been illustrated and described, it should be understood that other shapes and configuration of slots 50, 52 are also contemplated as falling within the scope of the invention.

In one embodiment of the invention, the distal end portion 32a of sleeve 32 is secured to the inner rod 30 by way of a compression ring 70. Specifically, the distal-most portion of sleeve 32 is disposed about portion 36 of rod 30, with the distal end of sleeve 32 abutting the shoulder 42 formed by the rounded distal end portion 38. The compression ring 70 is positioned about the distal-most portion of sleeve 32 and is compressed thereabout, such as, for example, by mechanical crimping to secure sleeve 32 to inner rod 30. As should be appreciated, slot portion 60 aids in tightly compressing sleeve 32 about inner rod 30 to provide secure engagement therebetween. It should be understood that compression ring 70 could alternatively be compressed about distal-most portion of sleeve 32 by other means, such as, for example, by forming compression ring 70 out of a shape-memory material that is reformable to a memorized configuration having an internal diameter that is less than the outer diameter of sleeve 32. It should further be understood that the distal-most end portion of sleeve 32 could be secured to rod 30 by other means, such as, for example, by fastening, welding, adhesion or other methods of attachment known to those of skill in the art.

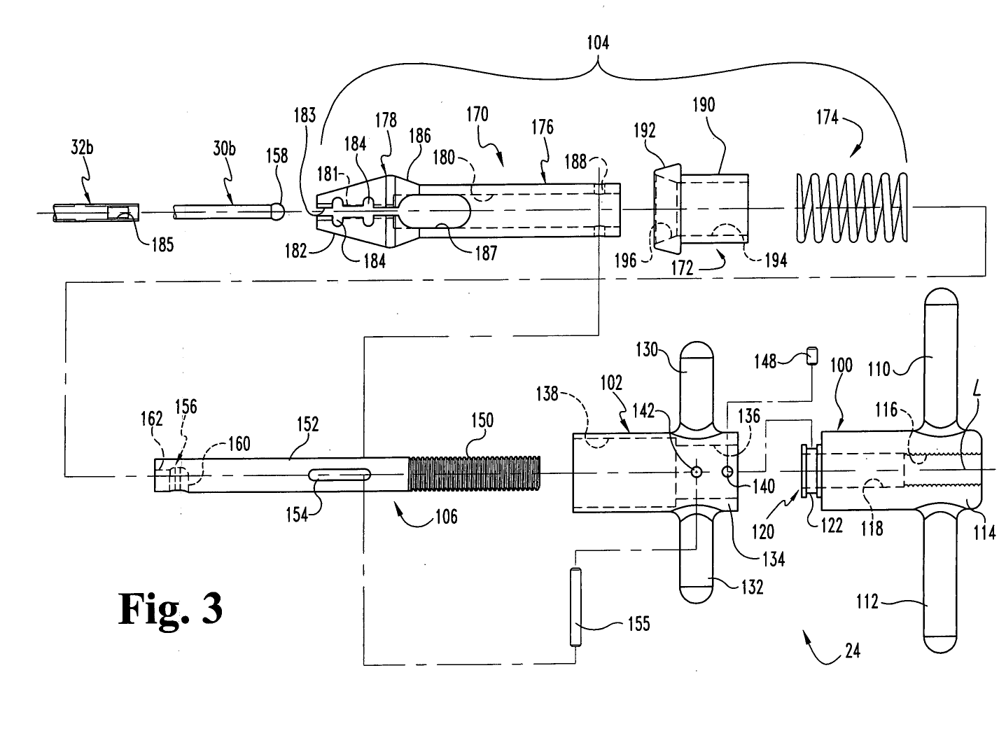

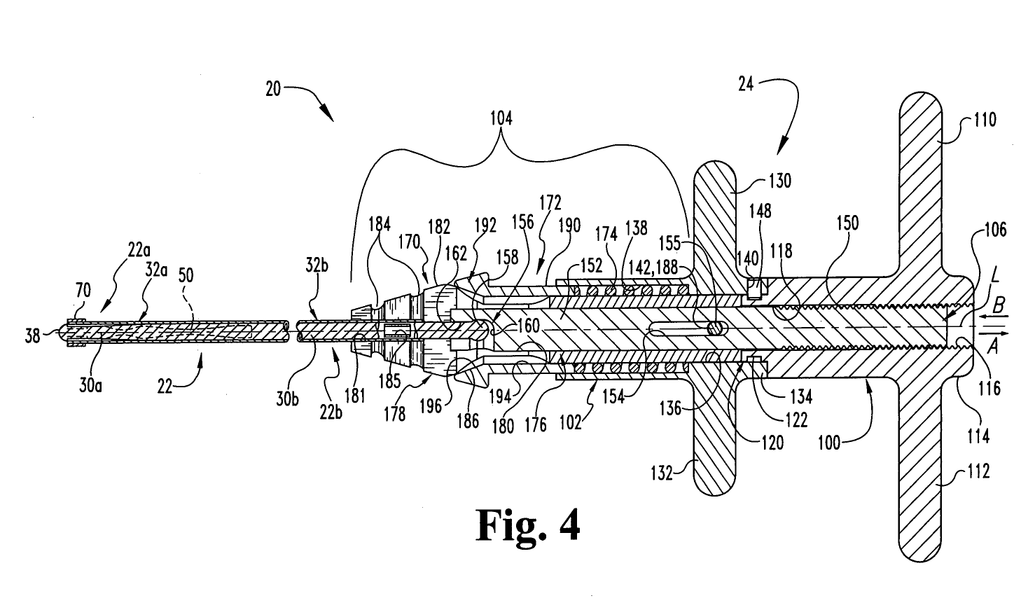

Referring to FIGS. 3 and 4, shown therein are further details regarding the actuator mechanism 24. Actuator mechanism 24 is generally comprised of a rotary handle 100, a stationary handle 102, a connector assembly 104, and an actuator member 106. As will be discussed in further detail below, the connector assembly 104 is configured to secure the elongate member 22, and more specifically the outer sleeve 32, to the remainder of the actuator mechanism 24. As will also be discussed below, the threaded actuator member 106 is coupled to the inner rod 30 and is engaged with the rotary handle 100 such that rotational displacement of handle 100 about longitudinal axis L linearly displaces the actuator member 106 along longitudinal axis L. As described above, the linear displacement of rod 30 relative to sleeve 32 causes the distal end portion 32a of sleeve 32 to reform from its initial configuration toward its deformed configuration.

The rotary handle 100 includes a pair of lateral extensions 110, 112 extending outwardly from a main body portion 114 to define a T-handle arrangement which aids the surgeon in rotating the handle 100 relative to the stationary handle 102. The main body portion 114 includes an opening extending along longitudinal axis L and having a threaded portion 116 and an unthreaded portion 118. A hub portion 120 extends from the main body portion 114 and defines an annular groove 122.

The stationary handle 102 includes a pair lateral extensions 130, 132 extending outwardly from a main body portion 134 to define a second T-handle arrangement which aids the surgeon in securely gripping instrument 20 and in maintaining the handle 102 in a stationary rotational position during rotation of handle 100. The main body portion 134 includes an opening extending therethrough along longitudinal axis L and defining a first cavity 136 and a second cavity 138. A pair of openings 140, 142 extend through the main body portion 134 and are disposed in communication with the first cavity 136. The hub portion 120 of handle 100 is inserted within the first cavity 136 and a pin or fastener 148 is inserted through opening 140 and positioned within the annular groove 122 to axially couple rotary handle 100 to stationary handle 102 while permitting relative rotational displacement therebetween.

The actuator member 106 includes a threaded shank portion 150 and an unthreaded shank portion 152. The threaded shank portion 150 is configured to threadingly engage the threaded opening 116 in rotary handle 100. In one embodiment of the invention, the threaded shank portion 150 and the threaded opening 116 each define right hand threads. The unthreaded shank portion 152 includes a slotted opening 154 extending therethrough that is aligned with the opening 142 in the stationary handle 102. A pin or fastener 155 is inserted through the opening 142 and the slotted opening 154 to couple the actuator member 106 to the stationary handle 102. As should be apparent, pin 155 substantially prevents relative rotational displacement between actuator member 106 and handle 102 while allowing a limited amount of relative linear displacement along longitudinal axis L. The distal end portion of the actuator member 106 includes a socket 156 configured to accept a corresponding ball portion 158 extending from the proximal end portion 30b of rod 30. The socket opening 156 includes a spherical portion 160 sized to receive the ball portion 158 therein, and a cylindrical portion 162 sized to receive the distal end portion 30b of rod 30 therethrough to connect rod 30 to actuator member 106. It should be understood, however, that other methods of interconnecting rod 30 and actuator member 106 are also contemplated as would occur to one of skill in the art.

As discussed above, the connector assembly 104 is configured to connect the elongate member 22, and more specifically the outer sleeve 32, to the remainder of the actuator mechanism 24. The connector assembly 104 is generally comprised of a gripper member 170, a lock collar member 172 and a biasing member 174. The gripper member 170 includes a connecting segment 176, a gripping segment 178 and a longitudinal passage having a first portion 180 extending through connecting segment 176 and a second portion 181 extending through the gripping segment 178. The first portion 180 of the passage is sized to receive the shank portion 152 of actuator member 150 therein, and the second portion 181 of the passage is sized to receive the proximal end portion 32b of sleeve 32 therein.

The gripping segment 178 of gripper member 170 has a generally conical shape and includes a tapered outer surface 182. The gripping segment 178 also includes a longitudinally extending slit 183 and a pair of transverse slots 184 that intersect slit 183, with both the slit 183 and the slots 184 intersecting the longitudinal passage 181. One purpose of the slit 183 and the slots 184 is to facilitate compression of the gripping segment 178 about the proximal end portion 32b of sleeve 32. The proximal end portion 32b of sleeve 32 defines an opening or window 185 extending therethrough to further facilitate gripping of sleeve 32 by gripping segment 178. Another purpose of slit 183 is to provide a passageway for the lateral insertion of the proximal end portion 30b of rod 30 therethrough to permit assembly with the actuator member 106. The gripping segment 178 also includes an outer tapered surface 186, the purpose of which will become evident below.

The connecting segment 176 of gripper member 170 defines an elongate opening 187 extending transversely therethrough and being positioned in communication with the longitudinal slit 183. One purpose of the elongate opening 187 is to facilitate compression of the gripping segment 178 about the proximal end portion 32b of sleeve 32. Another purpose of the transverse slot 187 is to provide a passageway for the lateral insertion of the ball portion 158 of rod 30 therethrough and into engagement with the socket 156 defined in actuator member 106. The connecting segment 176 also includes an opening 188 extending transversely therethrough and aligned with the opening 142 in the stationary handle 102. Pin 155 is inserted through the opening 188 to axially couple the gripper member 170, and in turn the elongate member 22, to the stationary handle 102 in a manner that substantially prevents relative linear and rotational displacement therebetween.

The lock collar member 172 includes a cylindrically-shaped body portion 190, a tapered end portion 192, and a longitudinal passage 194 extending therethrough and being sized to receive the connecting segment 176 of gripper member 170 therein. The cylindrical body portion 190 is sized to be received within cavity 138 of stationary handle 102. The longitudinal passage 194 includes an inner tapered surface 196 that corresponds to the outer tapered surface 186 of gripping segment 178. In one embodiment of the invention, the biasing member 174 is a coil spring. However, it should be understood that other types of biasing devices may alternatively be used as would occur to one of skill in the art.

Referring to FIG. 4, spring 174 is disposed within the cavity 138 of stationary handle 102 and is engaged against the proximal end of the lock collar 172 to bias the lock collar 172 toward the gripping segment 178. The biasing of lock collar 172 engages the tapered inner surface 196 tightly against the tapered outer surface 186 of gripping segment 178. Such engagement creates an inward compression force onto the gripping segment 178, which in turn causes the gripping segment 178 to collapse tightly about the proximal end portion 32b of sleeve 32 to securely grip sleeve 32 within the longitudinal passage 181. The tapered outer surface 192 of lock collar 172 is oriented at about the same angle as the tapered outer surface 182 of gripping segment 178 to provide a relatively smooth transition between lock collar 172 and gripping segment 178.

Based on the above description and corresponding illustrations, it should be apparent that rotation of handle 100 relative to stationary handle 102 in a clockwise direction (assuming right hand threading) will cause the actuator member 106 to be linearly displaced in the direction of arrow A, which will correspondingly cause rod 30 to be linearly displaced in the direction of arrow A. Furthermore, since the distal end portion of sleeve 32 is engaged with the distal end portion of rod 30, linear displacement of rod 30 in the direction of arrow A will cause the deformable distal end portion 32a of sleeve 32 to buckle outwardly toward the deformed configuration illustrated in FIG. 6. It should also be apparent that rotation of handle 100 relative to stationary handle 102 in a counter-clockwise direction will cause the actuator member 106 to be linearly displaced in the direction of arrow B, which will correspondingly cause rod 30 to be linearly displaced in the direction of arrow B. Linear displacement of rod 30 in the direction of arrow B will cause the deformable distal end portion 32a of sleeve 32 to reform back toward the insertion configuration illustrated in FIG. 5. As should be apparent, instead of rotating handle 100 relative to handle 102 to impart relative linear displacement between rod 30 and sleeve 32, it is also possible to hold handle 100 in a stationary position and to rotate handle 102 relative to handle 100 to impart relative linear displacement between rod 30 and sleeve 32.

Although one specific embodiment of the actuator mechanism 24 has been illustrated and described herein, it should be understood that the use of other types and configurations of actuator mechanisms are also contemplated as would occur to one of skill in the art. As should be apparent, any type of actuator mechanism that is capable of imparting relative displacement between rod 30 and sleeve 32 to reform the distal end portion 32a of sleeve 32 between the initial and deformed configurations may be used. It should further be understood that in an alternative form of the invention, rod 30 may be manually displaced by the surgeon relative to sleeve 32, thereby eliminating the need for a separate actuator mechanism 24.

Referring now to FIGS. 5 and 6, shown therein is the distal end portion 22a of elongate member 22, as shown in an initial insertion configuration and a mechanically deformed expanded configuration, respectively. When in the initial configuration (FIG. 5), the distal end portion 32a of sleeve 32 has a relatively low profile to facilitate positioning adjacent a vertebral body. As should be appreciated, the rounded distal end portion 38 reduces the likelihood of damage to adjacent tissue during such positioning. As used herein, positioning of the distal end portion 32a adjacent a vertebral body is meant to include positioning of the distal end portion 32a in proximity to a vertebral body, within a vertebral body or within a space between adjacent vertebral bodies. As discussed above, instrument 20 may also be used in association with spinal structures other than a vertebral body, such as, for example, a spinal implant, with the distal end portion 32a of sleeve 32 being positioned adjacent or within the spinal implant when in the insertion configuration.

Once properly positioned adjacent the vertebral body, the distal end portion 32a of sleeve 32 is mechanically deformed by displacing the rod 30 relative to the sleeve 32. In the illustrated embodiment of the invention, such relative displacement is accomplished by linearly displacing rod 30 relative to sleeve 32 in the direction of arrow A, and is initiated by the selective actuation of actuator mechanism 24. In an alternative embodiment of the invention, the distal end portion 32a of sleeve 32 may be mechanically deformed toward the expanded configuration by way of relative rotational displacement between rod 30 and sleeve 32.

When reformed toward the expanded configuration (FIG. 6), the distal end portion 32a of sleeve 32 is outwardly deformed relative to longitudinal axis L so as to form a number of laterally extending projections or protrusions 198a, 198b. As discussed above, the deformed configuration of instrument 20 may define any number of laterally extending projections, including a single projection or three or more projections, and may define a number of laterally extending projections at various axial locations along longitudinal axis L. It should be apparent that the number, position, and direction of the laterally extending projections is at least partially controlled by the configuration and placement of the slots 50 in sleeve 32. In this manner, formation of the laterally extending projections and the resulting preparation of the vertebral body is said to be directionally controlled. Moreover, if the deformed configuration of instrument 20 defines a single projection 198a, or a single pair of opposing projections 198a, 198b aligned along a common transverse axis T, then formation of the laterally extending projection and the resulting preparation of the vertebral body is said to be uniaxial. Further, if the deformed configuration of instrument 20 defines a single projection 198a extending in a single direction, then formation of the laterally extending projection and the resulting preparation of the vertebral body is said to be unidirectional.

Following preparation of the vertebral body, the distal end portion 32a of sleeve 32 may be reformed from its deformed/expanded configuration back toward its initial insertion configuration by linearly displacing rod 30 relative to sleeve 32 in the direction of arrow B. As discussed above, the distal end portion 32a of sleeve 32 may be formed of a shape-memory material, such as, for example, a shape-memory alloy (“SMA”) to aid in reforming the distal end portion 32a from the deformed configuration back toward its initial configuration. More specifically, SMAs are known to exhibit a characteristic or behavior in which a particular component formed of an SMA is capable of being deformed from an initial “memorized” shape or configuration to a different shape or configuration, and then reformed back toward its initial shape or configuration.

Further details regarding the superelastic phenomena of a SMA and additional characteristics of stress-induced martensite are more fully described by Yuichi Suzuki in an article entitled Shape Memory Effect and Super-Elasticity in Ni—Ti Alloys, Titanium and Zirconium, Vol. 30, No. 4, October 1982, the contents of which are hereby incorporated by reference. Additionally, while there are many alloys that exhibit shape-memory or superelastic characteristics, one of the more common SMAs is an alloy of nickel and titanium. One such well-known SMA is Nitinol. It should be understood, however, that other SMA materials that exhibit superelastic characteristics are contemplated as being within the scope of the invention.

If the distal end portion 32a of outer sleeve 32 is formed of an SMA material and is reshaped or deformed while at a temperature above the transformation temperature A, of the SMA, the distal end portion 32a will automatically recover or reform toward its initial shape or configuration when the stress is removed from distal end portion 32a. As illustrated in FIG. 5, when distal end portion 32a is in its unstressed initial configuration, virtually all of the SMA material will be in an austenitic state. However, upon the imposition of stress onto distal end portion 32a (e.g., by turning actuator handle 100 in a clockwise direction relative to stationary handle 102), at least a portion of the SMA material will transform into reversible stress-induced martensite as the distal end portion 32a is deformed toward the expanded configuration. Upon the reduction or removal of the stress (e.g., by turning actuator handle 100 in a counter clockwise direction), at least a portion of the SMA material will be transformed back into austenite and the distal end portion 32a will automatically reform back toward the initial configuration.

In some embodiments of the invention, the projections 198a, 198b may be designed to provide a cutting edge 55 that is exposed to cut tissue when the projections 198a, 198b are extended. The cutting edge 55 may be a thin portion of the sleeve 32, or in some embodiments may be sharpened to an edge that is significantly thinner than the thickness of the sleeve 32 to provide a sharper cutting edge. The cutting edge 55 may be tempered, serrated, or otherwise treated or configured to enhance the ability of the projections to cut through tissue, as is known in the art of tissue cutting devices.

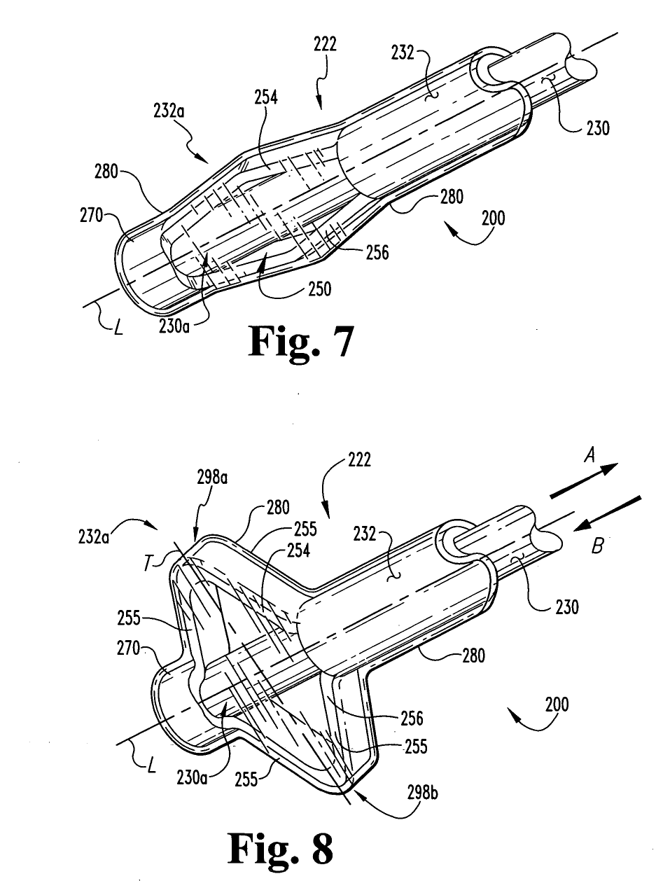

Referring now to FIGS. 7 and 8, shown therein is the distal end portion of an instrument 200 according to another form of the present invention, as shown in an initial insertion configuration and a mechanically deformed configuration, respectively. It should be understood that instrument 200 may be used in association with applications similar to those discussed above with regard to instrument 20, including both intrabody and interbody applications involving preparation or displacement of at least a portion of a vertebral body.

Instrument 200 is generally comprised of an elongate member 222 extending along a longitudinal axis L and having a distal end portion (as shown) and a proximal end portion (not shown) coupled to an actuator mechanism which may be configured similar to actuator mechanism 24. The distal end portion of elongate member 222 is deformable and is configured to outwardly expand in response to a mechanically induced force. Specifically, the distal end portion is reformable between an initial configuration (FIG. 7) for positioning adjacent a vertebral body, and a deformed configuration (FIG. 8) for preparation of at least a portion of the vertebral body. Although the illustrated embodiment depicts elongate member 222 as having a generally linear, unitary configuration, it should be understood that elongate member 222 may take on other configurations as well, such as, for example, a curvilinear configuration or a hinged configuration.

In the illustrated embodiment of instrument 200, the elongate member 222 is generally comprised of an inner rod member 230 and an outer sleeve member 232. The inner rod 230 may be formed of a substantially rigid medical grade material such as, for example, titanium or stainless steel. The rod 230 includes a distal end portion 230a that is disposed within and coupled to a distal end portion 232a of sleeve 232. Although rod 230 has been illustrated and described as having a substantially circular cross, it should be understood that other shapes and configurations are also contemplated as being within the scope of the present invention, such as, for example, elliptical, square, rectangular or other polygonal configurations.

The outer sleeve 232 illustrated has a tubular configuration defining an inner passage extending therethrough generally along longitudinal axis L and sized to slidably receive rod 230 therein. Sleeve 232 is formed of a relatively flexible material that is capable of being reformed from an initial configuration to an expanded configuration. The sleeve 232 may be formed of a relatively elastic material that is capable of being elastically deformed to the expanded configuration and reformed back toward the initial configuration. Sleeve 232 may be formed of materials including, but not limited to, titanium, stainless steel, an elastomer, a polymer, a rubber, a composite material or a shape-memory material. Although the entire length of sleeve 232 may be formed of a flexible, elastic material, it should be understood that only the distal end portion 232a need be formed of such material, with the remainder of sleeve 232 being formed of any suitable medical grade material. Additionally, although sleeve 232 is illustrated as having a substantially cylindrical or tubular configuration, it should be understood that other shapes and configurations of sleeve 232 are also contemplated as being within the scope of the present invention. Furthermore, although sleeve 232 has been illustrated and described as being formed as a single-piece, unitary structure, it should be understood that the distal end portion 232a could be formed separately from the remainder of sleeve 232, and coupled together by any known method, such as, for example, by fastening, welding or adhesion.

In one embodiment of instrument 200, the distal-most end portion 270 of sleeve 232 is secured to the distal end portion 230a of rod 230 by way of crimping. In other embodiments, sleeve portion 270 may be connected to rod portion 230a by a compression ring similar to compression ring 70, or by other connection techniques such as, for example, fastening, welding, adhesion, or other methods of attachment known to those of skill in the art.

The distal end portion 232a of sleeve 232 includes at least one rectangular-shaped window or slot 250 extending generally along longitudinal axis L, and may include at least a pair of slots 250 and 252 (not shown) disposed generally opposite one another so as to define a pair of longitudinally extending flexible strips of material 254, 256. However, it should be understood that the distal end portion 232a of sleeve 232 could define any number of longitudinally extending slots, including three or more slots, which would in turn define a corresponding number of flexible strips of material disposed between the slots. The slots 250, 252 are provided to facilitate outward buckling of the distal end portion 232a of sleeve 232 upon the imposition of relative linear displacement between rod 230 and sleeve 232. As illustrated in FIG. 8, when reformed toward the expanded configuration, the flexible strips of material 254, 256 will outwardly buckle along transverse axis T at a location adjacent the midpoint of slots 250, 252. In the illustrated embodiment of instrument 200, the slots 250, 252 are substantially identical in shape and configuration. However, it should be understood that slots 250, 252 may take on different predetermined shapes and configurations. Additionally, although slots 250, 252 and strips of material 254, 256 are illustrated as having a generally rectangular shape, other predetermined shapes and configurations are also contemplated.

When in the initial configuration (FIG. 7), the distal end portion 232a of sleeve 232 has a relatively low profile to facilitate positioning adjacent a vertebral body. However, once properly positioned adjacent the vertebral body, the distal end portion 232a is mechanically deformed by displacing rod 230 relative to sleeve 232. In the illustrated embodiment, such relative displacement is accomplished by linearly displacing rod 230 relative to sleeve 232 in the direction of arrow A. In an alternative form of the present invention, the distal end portion 232a of sleeve 232 may be mechanically deformed toward the expanded configuration by way of relative rotational displacement between rod 230 and sleeve 232.

When reformed toward the expanded configuration (FIG. 8), the distal end portion 232a of sleeve 232 is outwardly deformed relative to longitudinal axis L so as to form a number of laterally extending projections or protrusions 298a, 298b. As discussed above, the deformed/expanded configuration of instrument 200 may alternatively define any number of laterally extending projections, including a single projection or three or more projections. Similar to instrument 20, formation of the laterally extending projections and the resulting preparation of the vertebral body by instrument 200 is directionally-controlled, and can be uniaxial, unidirectional or both uniaxial and unidirectional. Following preparation of the vertebral body, the distal end portion 232a of sleeve 232 may be reformed back toward its initial insertion configuration by linearly displacing rod 230 relative to sleeve 232 in the direction of arrow B. As discussed above with regard to instrument 20, the distal end portion 232a of sleeve 232 may be formed of a shape-memory material, such as, for example, a shape-memory alloy to aid in reforming distal end portion 232a back toward its initial configuration.

In some embodiments of the invention, the projections 298a, 298b may be designed to provide a cutting edge 255 that is exposed to cut tissue when the projections 298a, 298b are extended. The cutting edge 255 may be a thin portion of the sleeve 232, or in some embodiments may be sharpened to an edge that is significantly thinner than the thickness of the sleeve 232 to provide a sharper cutting edge. The cutting edge may be tempered, serrated, or otherwise treated or configured to enhance the ability of the projections to cut through tissue, as is known in the art of tissue cutting devices.

In one embodiment of the invention, at least the distal end portion of the elongate member 222 is covered by a flexible membrane 280. The flexible membrane 280 may be formed of a resilient material that is capable of conforming to the shape of the distal end portion 232a of sleeve 232 during reformation between the initial and deformed configurations. Such flexible materials include, but are not limited to, silicone, latex, rubber, a polymer or other suitable elastomeric materials. One purpose of the flexible membrane 280 is to prevent tissue or other foreign material from passing through the slots 250, 252 and being deposited within the space between the strips of material 254, 256 and the rod 230 and/or between the rod 230 and the remainder of the sleeve 232. As should be appreciated, such a build-up of tissue or foreign material may block or otherwise inhibit reformation of the distal end portion 232a of sleeve 232 from the deformed configuration (FIG. 8) back toward the initial configuration (FIG. 7). Although the flexible membrane 280 is illustrated as covering the distal end portion of elongate member 222, it should be understood that the flexible membrane 280 could be sized to cover the entire length of the elongate member 222. It should also be understood that a flexible membrane similar to flexible membrane 280 may be used in association with the surgical instrument 20 discussed above and/or the surgical instrument 300 discussed below.

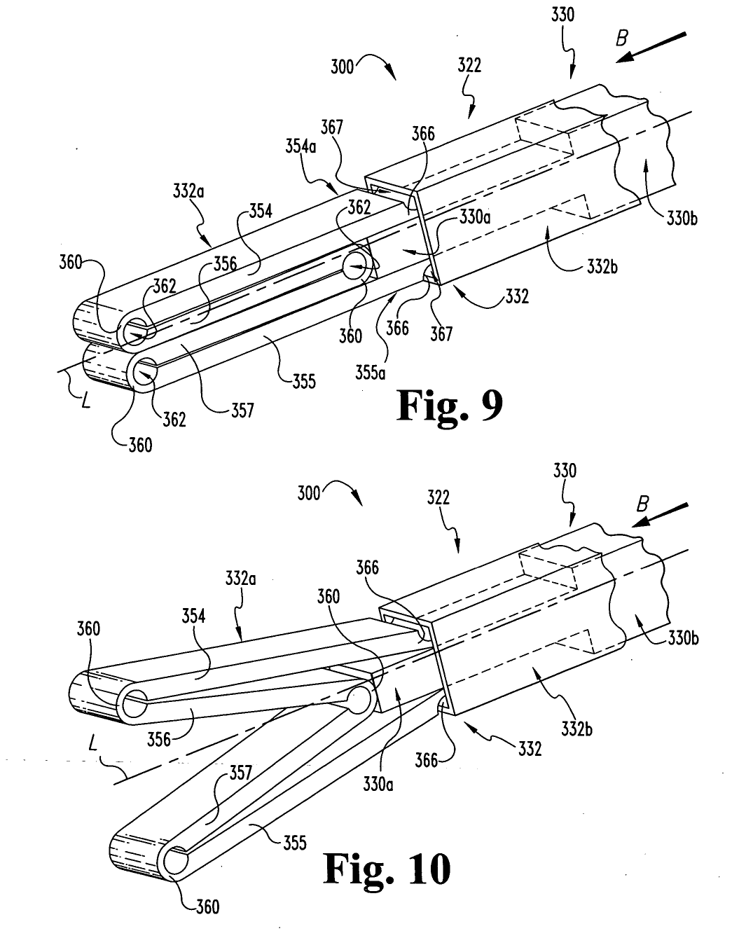

Referring now to FIGS. 9-11, shown therein is the distal end portion of an instrument 300 according to another form of the present invention, as shown in an initial insertion configuration, a partially deformed intermediate configuration, and a fully deformed configuration, respectively. It should be understood that instrument 300 may be used in association with applications similar to those discussed above with regard to instrument 20, including both intrabody and interbody applications involving preparation or displacement of at least a portion of a vertebral body.

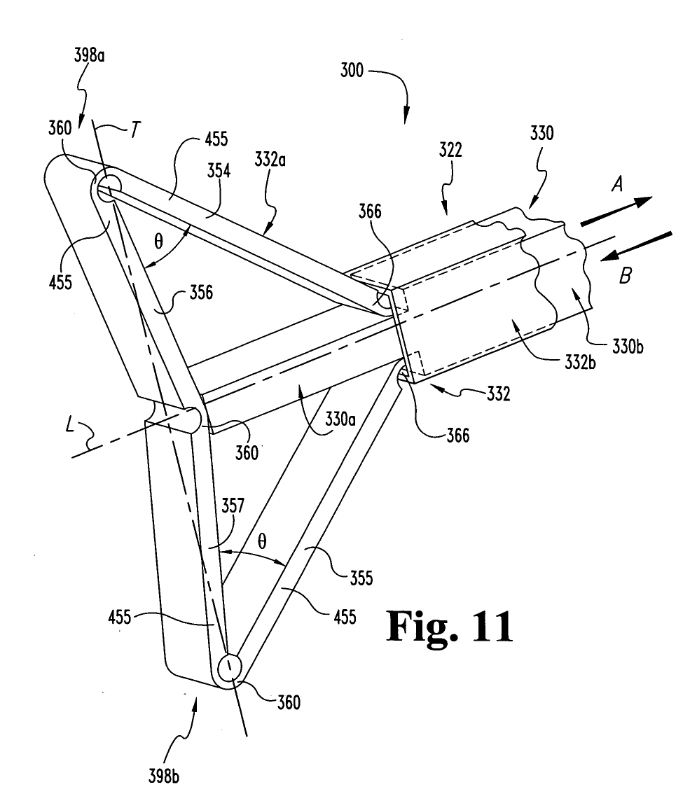

Instrument 300 is comprised of an elongate member 322 extending generally along a longitudinal axis L and having a distal end portion (as shown) and a proximal end portion (not shown) which may be coupled to an actuator mechanism similar to actuator mechanism. The distal end portion is deformable and is configured to outwardly expand upon the imposition of a mechanically induced force. Specifically, the distal end portion is reformable between an initial configuration (FIG. 9) for positioning adjacent a vertebral body, and a deformed configuration (FIG. 11) for preparation of at least a portion of the vertebral body. Although the illustrated embodiment depicts elongate member 322 as having a generally linear, unitary configuration, it should be understood that elongate member 322 may take on other configurations as well, such as, for example, a curvilinear configuration or a hinged configuration.

In the illustrated embodiment of instrument 300, the elongate member 322 is generally comprised of an inner rod member 330 and an outer sleeve member 332. The inner rod 330 may be formed of a substantially rigid medical grade material such as, for example, titanium or stainless steel. Rod 330 includes a distal end portion 330a extending from a main body portion 330b. In the illustrated embodiment, the distal end portion 330a has a rectangular shape and the main body portion 330b has a square shape. However, it should be understood that other shapes and configurations of rod 330 are also contemplated as being within the scope of the present invention such as, for example, circular, elliptical or polygonal configurations.

The outer sleeve 332 has a deformable distal end portion 332a coupled to a main body portion 332b. The main body portion 332b has a square configuration defining an inner passage extending therethrough generally along longitudinal axis L and sized to slidably receive portion 330b of rod 330 therein. However, it should be understood that other shapes and configurations of sleeve portion 332b are also contemplated as being within the scope of the present invention. The main body portion 332b shown is formed of a substantially rigid material, such as, for example, titanium, stainless steel or other substantially rigid medical grade materials.

The deformable distal end portion 332a of sleeve 332 is at least partially formed of a relatively flexible material that is capable of being reformed from the initial configuration illustrated in FIG. 9 toward the deformed configuration illustrated in FIG. 11. In some embodiments, the distal end portion 332b is formed of a relatively elastic material that is capable of being elastically deformed toward the deformed configuration and reformed back toward the initial configuration. The deformable distal end portion 332b may be formed of materials including, but not limited to, titanium, stainless steel, an elastomer, a polymer, a rubber, a composite material or a shape-memory material. Distal end portion 332b shown is formed separately from main body portion 332a and connected thereto by any method know to one of skill in the art, such as, for example, by fastening, welding or adhesion. However, is should be understood that distal end portion 332b could alternatively be formed integral with main body portion 332a to define a single-piece, unitary structure.

The deformable distal end portion 332a of sleeve 332 includes a plurality of wall elements 354-357 that are flexibly interconnected by a number or interconnection portions 360. In one embodiment of the invention, the interconnection portions 360 are defined by forming an opening or channel 362 at locations where adjacent wall elements adjoin to one another. In one embodiment of the invention, the wall elements 354-357 are integrally formed to define a unitary, single-piece reformable structure that is collapsible to define a relatively low-profile insertion configuration and expandable to define an outwardly deformed configuration.

To aid in reformation of the distal end portion 332a between the insertion and deformed configurations, the distal end portion 332a of sleeve 332 may be flexibly coupled to the main body portion 332b. In one embodiment, the outer wall elements 354, 355 each include a flexible interconnection portion 366 defined by forming an opening or channel 367 adjacent their respective distal end portions 354a, 355a. The distal end portions 354a, 355a of the outer wall elements 354, 355 are in turn coupled to inner surfaces of the main body portion 332b of sleeve 332, such as, for example, by fastening, welding or adhesion. The outer wall elements 354, 355 are separated by a distance sufficient to receive the distal end portion 330a of rod 330 therebetween.

As shown in FIG. 9, the insertion configuration has a substantially rectangular-shaped profile, with each of the wall elements 354-357 being disposed in a substantially uniform orientation (i.e., parallel to one another), and with the two inner wall elements 356, 357 being disposed between the two outer wall elements 354, 355. As shown in FIG. 11, the deformed/expanded configuration has a substantially triangular-shaped profile, with the two inner wall elements 356, 357 being disposed in a substantially parallel and co-linear orientation, and the two outer wall elements 354, 355 being disposed at an angle e relative to inner wall elements 356, 357. In one embodiment, the angle θ is about 30°-45°. It should be understood that other insertion and expanded configurations are also contemplated as falling within the scope of the present invention. Additionally, although the reformable distal end portion 332b of sleeve 332 has been illustrated and described as including four wall elements 354-357, it should be understood that any number of wall elements may be flexibly interconnected to form the refofrmable distal end portion 332b.

When in the initial folded configuration illustrated in FIG. 9, the deformable distal end portion 332a of sleeve 332 has a relatively low profile to facilitate positioning adjacent a vertebral body. However, once properly positioned adjacent the vertebral body, the distal end portion 332a is mechanically deformed by displacing rod 330 relative to sleeve 332. In the illustrated embodiment, such relative displacement is accomplished by linearly displacing rod 330 relative to sleeve 332 in the direction of arrow B, and is initiated by the selective actuation of an actuator mechanism (not shown).

As shown in FIG. 10, relative displacement of rod 330 in the direction of arrow B causes the distal end portion 330a of rod 330 to engage the interconnection portion 360 extending between the inner wall elements 356, 357, thereby initiating the outward expansion or unfolding of the wall elements 354-357. In one embodiment of the invention, the distal end portion 330a of rod 330 is secured to the interconnection portion 360, such as, for example, by fastening, welding or adhesion. However, it should be understood that the distal end portion 330a of rod 330 need not necessarily be rigidly secured to interconnection portion 360, but could alternatively form an abutting relationship therewith to initiate the outward expansion of wall elements 354-357.

As shown in FIG. 11, when reformed to the deformed configuration, the wall elements 354-357 are unfolded and expanded outwardly relative to longitudinal axis L so as to form laterally extending projections or protrusions 398a, 398b disposed along a transverse axis T. Although instrument 300 has been illustrated and described as including a pair of oppositely disposed projections 398a, 398b when in the expanded configuration, it should be understood that the distal end portion 332a of sleeve 332 may be configured to define any number of projections, including a single projection or three or more projections. Further, similar to instrument 20, the expansion of the distal end portion 332a of sleeve 332 and the resulting preparation of the spinal structure accomplished by instrument 300 is directionally-controlled, and can be uniaxial, unidirectional or both uniaxial and unidirectional.

In some embodiments of the invention, the wall elements 354-357 may be designed to provide cutting edges 455 that are exposed to cut tissue when the wall elements 354-357 are extended. The cutting edges 455 may be essentially the same thickness as wall elements 354-357, or in some embodiments may be sharpened to an edge that is significantly thinner than the thickness of the wall elements 354-357 to provide a sharper cutting edge. The cutting edge may be tempered, serrated, or otherwise treated or configured to enhance the ability of the projections to cut through tissue, as is known in the art of tissue cutting devices.

Following preparation of the vertebral body, the distal end portion 332a of sleeve 332 may be reformed toward its initial insertion configuration by linearly displacing rod 330 relative to sleeve 332 in the direction of arrow A (FIG. 11). As discussed above with regard to instrument 20, the distal end portion 332a of sleeve 332 may be formed of a shape-memory material, such as, for example, a shape-memory alloy (“SMA”) to aid in reforming distal end portion 332a back toward its initial configuration.

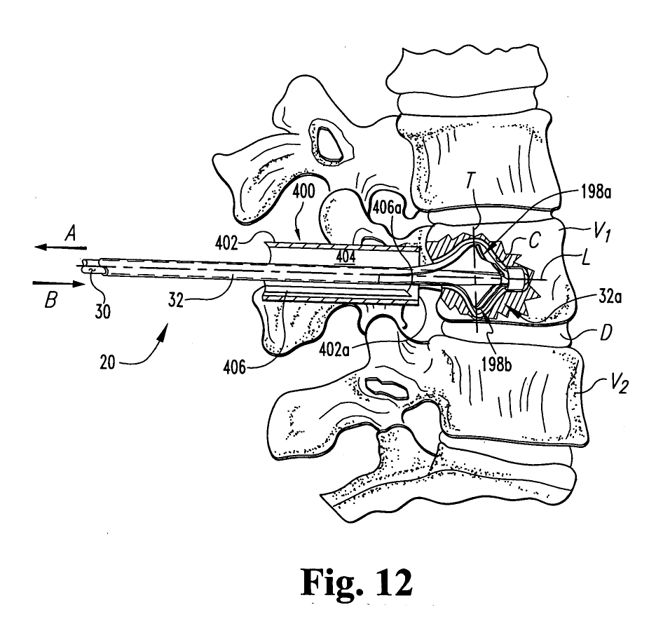

Referring to FIG. 12, shown therein is a lateral view of a spinal column, illustrating the introduction and expansion of instrument 20 within a vertebral body V₁ to perform intrabody distraction. The distal end portion 32a of sleeve 30 is initially passed through an access opening (not shown) extending through an outer wall of the vertebral body V₁ while in the undeformed initial configuration illustrated in FIG. 5. Subsequent to insertion within the vertebral body V₁, the distal end portion 32a of sleeve 32 is reformed by a mechanically-induced force created by linearly displacing rod 30 relative to sleeve 32 in the direction of arrow A. As a result, the distal end portion 32a is outwardly deformed to form opposing projections 198a, 198b extending along transverse axis T. Such outward deformation is particularly useful, for example, to compact or compress cancellous bone against the inner cortical wall of the vertebral body V₁ to form a cavity C therein. Compaction of the cancellous bone may have the effect of exerting an outward force on the inner surface of the cortical wall, making it possible to elevate or push broken and/or compressed bone back to or near its original pre-fracture condition or another desired condition. Alternatively, the opposing projections 198a, 198b may bear directly against the inner surface of the cortical bone to reduce a compression fracture in the vertebral body V₁.

In one form of the present invention, access into the inner cancellous region of the vertebral body V₁ is be accomplished by drilling a relatively small access opening through an outer wall of the vertebral body, such as, for example, through the pedicular region of the vertebral body V₁. The undeformed initial configuration of the distal end portion 32a of sleeve 30 is sized to pass through the small access opening to gain access to the inner cancellous region of the vertebral body V₁. In this manner, insertion of the distal end portion 32a of sleeve 32 is accomplished in a minimally invasive manner. Additionally, unlike certain prior art devices that require a relatively larger access opening to accommodate spreading of the proximal end portions of opposing members attached to one another in a scissors-like manner, only the distal end portion 32a of sleeve 32 is outwardly expanded when reformed toward the deformed configuration.

In one embodiment of the invention, the initial configuration of the distal end portion 32a of sleeve 32 is sized to pass through an access opening having a diameter between about 1 millimeter and about 5 millimeters. In a specific embodiment, the initial configuration of the distal end portion 32a is sized to pass through an access opening having a diameter of about 3 millimeters. In another embodiment of the invention, the deformed configuration of the distal end portion 32a of sleeve 30 is sized to displace the vertebral body V₁ within a range of about 3 millimeters to about 15 millimeters. In a specific embodiment, the deformed configuration of the distal end portion 32a is sized to displace the vertebral body V₁ about 10 millimeters. In another specific embodiment of the invention, the instrument 20 is capable of assuming a deformed configuration that is over three times greater than its initial configuration. Although ranges and specific sizes of the initial and deformed configurations of distal end potion 32b of sleeve 32 have been set forth above, it should be understood that such ranges and specific sizes are exemplary and are not intended to limit the scope of the present invention in any manner whatsoever.

Following preparation of the vertebral body V₁, the distal end portion 32a of sleeve 32 is reformed toward its initial insertion configuration by displacing rod 30 relative to sleeve 32 in the direction of arrow B. As a result, the opposing projections 198a, 198b are inwardly deformed to the extent necessary to provide uninhibited removal of the distal end portion 32a of sleeve 32 from the vertebral body V₁. As discussed above, reformation of the instrument 20 back toward its initial insertion configuration may be facilitated by forming the distal end portion 32a of sleeve 32 from a shape-memory material. Following the removal of instrument 20 from the vertebral body V₁, the cavity C may be filled with a biocompatible filling material, such as, for example, methylmethacrylate cement (e.g., bone cement), a structural implant, and/or a therapeutic substance to promote healing. Once set to a hardened condition, the filling material provides internal structural support to the vertebral body V₁, and more particularly provides structural support to the cortical bone of the vertebral body V₁.

In another form of the present invention, a cannula assembly 400 may be used to provide minimally invasive access to the vertebral bodies V₁, V₂ and/or the disc space D. As shown in FIG. 12, use of the cannula assembly 400 permits preparation of the vertebral body V₁ via insertion and manipulation of instrument 20 through a single working channel. Further details regarding a cannula assembly suitable for use in association with the present invention are disclosed in U.S. Pat. No. 6,599,291 to Foley et al., filed on Oct. 20, 2000, the contents of which are incorporated herein by reference.

The cannula assembly 400 includes a cannula 402 having a distal end 402a and defining an inner working channel 404 extending between the distal end 402a and a proximal end (not shown). The length of the cannula 402 is sized such that the proximal end (not shown) of the cannula 402 is positioned beyond the skin of the patient when the distal end 402a is positioned adjacent the vertebral body V₁. One advantageous feature of the cannula assembly 400 is the relatively large cross section of the working channel 404 extending through cannula 402. Such a large cross section permits the surgeon to introduce a wide variety of instruments or tools into the working channel 404, as well as the simultaneous introduction of two or more instruments or tools. Furthermore, the relatively large cross section of working channel 404 permits a wide range of motion of the instruments and tools.

The cannula assembly 400 may also include an endoscope assembly (not shown) mounted to the proximal end portion of the cannula 402 to provide remote visualization of the surgical site. The endoscope assembly may include, for example, a viewing element 406 disposed within the working channel 404 of cannula 402 and having a distal end 406a positioned adjacent the surgical site. The viewing element 406 in some embodiments is linearly and rotatably displaceable within the working channel 404 to provide a wide degree of visualization of the surgical site. The endoscope assembly may also include an illumination element (not shown), a remote viewing apparatus such as an eyepiece (not shown), and/or irrigation and aspiration components (not shown) extending along viewing element 406. One embodiment of an endoscope assembly suitable for use in association with the present invention is described in U.S. Pat. No. 6,152,871 to Foley et al., issued on Nov. 28, 2000, the contents of which are incorporated herein by reference. The cannula assembly 400 may also include a microscopic viewing system (not shown) mounted to the proximal end portion of the cannula 402 to provide microscopic visualization of the surgical site. One embodiment of a microscopic viewing system suitable for use in association with the present invention is described in U.S. Pat. No. 6,679,833 to Foley et al., filed on Mar. 23, 2001, the contents of which are incorporated herein by reference.

Although FIG. 12 illustrates the use of instrument 20 to at least partially displace the vertebral body V₁, it should be understood that instruments 200 and 300 could alternatively be used to perform the technique. It should also be understood that in addition to performing intrabody distraction, instruments 20, 200 and 300 may be used to perform interbody distraction of one or both of the adjacent vertebral bodies V₁, V₂, such as, for example, to increase the height of the disc space D. Interbody distraction of adjacent vertebral bodies V₁, V₂ may also be effective to increase the distance between corresponding portions of the vertebral bodies V₁, V₂. In cases involving brittle portions of the vertebral bodies V₁, V₂, shims may be positioned between the deformable distal end portion 32a of sleeve 32 and the vertebral bodies V₁, V₂ to distribute the compressive force over a larger area to avoid puncturing or crushing of the brittle portions. It should additionally be understood that although the distraction technique illustrated in FIG. 12 uses a posterior surgical approach, other surgical approaches are also contemplated, such as, for example, anterior, lateral, and postero-lateral approaches.

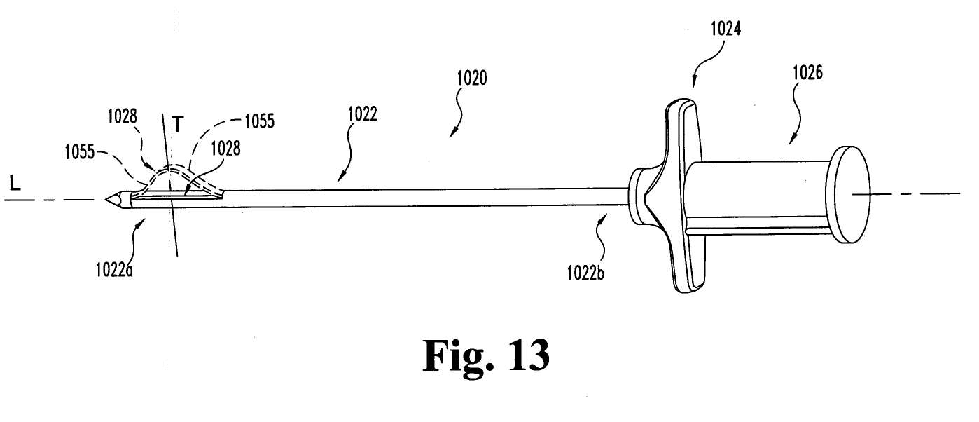

Referring to FIG. 13, shown therein is another embodiment of an instrument 1020 for treatment of the spine according to one form of the present invention. The illustrated instrument 1020 is designed for planned disposal upon use in association with a limited number of surgical procedures. In a specific embodiment, the instrument 1020 is designed for a single use in association with a single surgical procedure. In instances where the instrument 1020 is designed for a single use, immediate disposal eliminates the requirements and costs associated with cleaning, sterilizing, repackaging, and/or storing the instrument 1020 for repeat use. However, it should be understood that the instrument 1020 may be designed for use in association with multiple surgical procedures or may be designed to have a predetermined life span for use in association with a predetermined number of spinal surgeries after which the instrument 1020 is subjected to disposal. The instrument 1020 is generally comprised of an elongate member 1022, a handle portion 1024, an actuator mechanism 1026, and a deformable portion 1028 that is selectively transitionable between an initial configuration (shown in solid lines) and a deformed configuration (shown in phantom lines).

The elongate member 1022 extends generally along a longitudinal axis L and has a distal portion 1022a and a proximal portion 1022b. Although the illustrated embodiment depicts the elongate member 1022 as having a generally linear, unitary configuration, it should be understood that elongate member 1022 may take on other configurations such as, for example, a curvilinear configuration or a hinged configuration. The handle portion 1024 aids in the manipulation and handling of the instrument 1020 and also includes a mechanism for connecting to a material delivery system, the detail of which will be discussed below. The actuator mechanism 1026 serves to transition the deformable portion 1028 between the initial and deformed configurations. The deformable portion 1028 is located adjacent the distal portion 1022a of the elongate member 1022 and outwardly expands along a transverse axis T in response to a mechanically induced force that is provided via selective actuation of the actuator mechanism 1026.

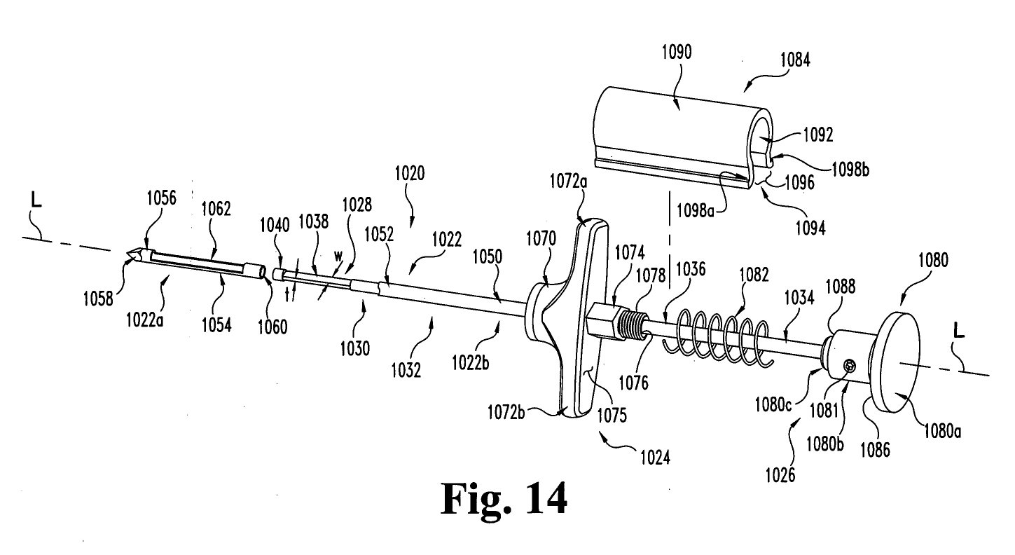

Referring to FIG. 14, shown therein is an exploded view of the instrument 1020 which illustrates additional elements and features associated with the elongate member 1022, the handled portion 1024, the actuator mechanism 1026 and the deformable portion 1028. Each of these components will now be discussed in greater detail.

In one embodiment of the invention, the elongate member 1022 is generally comprised of an inner rod member 1030 and an outer sleeve member 1032. The inner rod 1030 includes a proximal end portion 1034, a main body portion 1036, a deformable distal portion 1038 (comprising the deformable portion 1028), and a distal end portion 1040. In one embodiment, the inner rod 1030 is formed as a single-piece, unitary structure. However, it should be understood that portions of the inner rod 1030 (such as the deformable portion 1038 and/or the distal end portion 1040) could be formed separately and coupled together by any known method such as by fastening, welding or adhesion.

In the illustrated embodiment, the proximal end portion 1034, the main body portion 1036 and the distal end portion 1040 have a generally circular outer cross section that substantially corresponds to the inner cross section of the outer sleeve 1032. However, it should be understood that other shapes and configurations are also contemplated as falling within the scope of the invention including, for example, elliptical, square, rectangular, hexagonal, or other arcuate or polygonal configurations. In the illustrated embodiment, the deformable portion 1038 comprises a relatively thin, flexible strip of material extending generally along the longitudinal axis L. In a specific embodiment, the deformable strip 1038 comprises a generally flat, spring-like element to facilitate transitioning between a relatively straight initial configuration and an outwardly deformed or buckled configuration. However, it should be understood that other suitable configurations of the deformable strip 1038 are also contemplated to facilitate transitioning between an initial configuration and an outwardly deformed configuration.

The inner rod 1030 may be formed of a medical grade material such as, for example, titanium or stainless steel. However, it should be understood that the inner rod 1030 may be formed of other suitable medical grade materials. For example, in one embodiment, the deformable strip 1038 may be formed of a flexible material that is capable of facilitating elastic deformation from the initial configuration toward the deformed configuration and reformation back toward the initial configuration. In a specific embodiment, at least the deformable strip 1038 is formed of a thin metallic material such as titanium or stainless steel, an elastomeric material, a polymeric material, a rubber material, a composite material, or any other suitable flexible material to facilitate transitioning of the deformable strip 1038 between the initial and deformed configurations. In another specific embodiment, at least the deformable strip 1038 may be formed of a shape-memory material exhibiting superelastic characteristics to facilitate transitioning of the deformable strip 1038 from the initial configuration to the deformed configurations and reformation back toward the initial configuration. For example, at least the deformable strip 1038 may be formed of a shape-memory alloy (“SMA”) such as, for example, Nitinol. As should be appreciated, the width, thickness, shape and/or cross section of the deformable strip 1038 have an effect on the deformation characteristics and each provide a degree of control over the outward deformation/buckling of the deformable strip 1038. Although the deformable strip 1038 is illustrated as having a having a generally rectangular axial cross section defining a substantially uniform width w, it should be understood that the deformable strip 1038 may define a non-uniform width w. For example, in an alternative embodiment, the deformable portion may define one or more cut-ins or grooves along its axial length. In a specific embodiment, the deformable strip 1038 may be configured to have an hour-glass configuration to provide predetermined deformation characteristics associated with outward expansion of the deformable strip 1038 along the transverse axis T. As should be appreciated, segments of the deformable strip 1038 having a reduced width w would tend to provide less resistance to bending and serve as flexion points to facilitate outward deformation/buckling adjacent the areas of reduced width. Additionally, although the deformable strip 1038 is illustrated as having a having a substantially uniform thickness t, it should be understood that the deformable strip 1038 may define a non-uniform thickness t to provide predetermined deformation characteristics associated with outward expansion of the deformable strip 1038 along the transverse axis T. As should be appreciated, segments of the deformable portion 1038 having a reduced thickness t would tend to provide less resistance to bending and would thereby facilitate outward buckling adjacent the areas of reduced thickness.

In some embodiments of the invention, the deformable strip 1038 may be designed to provide a cutting edge 1055 that is exposed to cut tissue when the deformable strip 1038 is extended. The cutting edge 1055 may be a thin portion of the deformable strip 1038, or in some embodiments may be sharpened to an edge that is significantly thinner than the thickness t of the deformable strip 1038 to provide a sharper cutting edge. The cutting edge may be tempered, serrated, or otherwise treated or configured to enhance the ability of the projections to cut through tissue, as is known in the art of tissue cutting devices.

In the illustrated embodiment of the invention, the inner rod 1030 includes a single deformable strip 1038 extending along the longitudinal axis L which is configured to outwardly deform/buckle in a single direction along the transverse axis T so as to provide controlled unidirectional expansion. However, it should be understood that in other embodiments of the invention, the inner rod 1030 may include two or more deformable strips of material 1038 extending along the longitudinal axis L which are configured to outwardly deform/buckle in multiple directions. In a specific embodiment, such outward deformation of the multiple strips of material would be limited to expansion along the transverse axis T so as to provide controlled uniaxial expansion.

The outer sleeve 1032 generally includes a proximal end portion 1050, a main body portion 1052, a distal portion 1054, and a distal end portion 1056. In the illustrated embodiment, the proximal end portion 1050 of the sleeve 1032 extends axially from the handle portion 1024 and the distal end portion 1056 defines a pointed tip or trocar 1058 to facilitate insertion into and/or through vertebral tissue. However, other configurations of the distal end portion 1056 are also contemplated such as, for example, configurations defining a blunt or rounded tip to provide non-traumatic passage through vertebral tissue. The outer sleeve 1032 shown is formed of a substantially rigid medical grade material such as, for example, titanium or stainless steel. However, it should be understood that the outer sleeve 1032 may be formed of other suitable medical grade materials.

In the illustrated embodiment of the invention, the outer sleeve 1032 has a tubular configuration defining an axial cannula passage 1060 extending generally along the longitudinal axis L and sized to slidably receive the inner rod 1030 therein, the purpose of which will be discussed below. In one embodiment, the cannula passage 1060 has a generally circular inner cross section substantially corresponding to the outer cross section of the main body portion 1036 and distal end portion 1040 of the inner rod 1030. However, it should be understood that other shapes and configurations are also contemplated as falling within the scope of the invention including, for example, elliptical, square, rectangular, hexagonal or other arcuate or polygonal configurations. Additionally, although the outer sleeve 1032 is illustrated as being formed as a single-piece, unitary structure, it should be understood that the distal end portion 1056 could be formed separately from the remainder of sleeve 1032 and coupled together by any known method such as by fastening, welding or adhesion.

In the illustrated embodiment of the invention, the distal portion 1054 of the outer sleeve 1032 defines a slotted opening 1062 extending transversely through the sidewall of the sleeve 1032 and communicating with the axial cannula passage 1060. The slotted opening 1062 is sized and shaped to receive the deformable portion 1038 of the inner rod 1030 therethrough when transitioned to the outwardly deformed configuration. Although the outer sleeve 1032 is illustrated as including a single slotted opening 1062, it should be understood that the outer sleeve 1032 may define any number of slotted openings for receiving a corresponding number of deformable portions associated with the inner rod 1030.

As discussed above, the handle portion 1024 aids in the manipulation and handling of the instrument 1020 and also includes a mechanism for connecting to a material delivery system. In one embodiment, the handle portion 1024 is generally comprised of a base portion 1070, a pair of lateral extensions 1072a, 1072b extending outwardly from the base portion 1070, and a connector portion 1074 extending proximally from the base portion 1070 in an axial direction. The handle portion 1024 also includes an axial passage 1076 extending through the base portion 1070 and the connector portion 1074, the purpose of which will be discussed below.

The outer sleeve 1032 extends distally from the base portion 1070 with the cannula passage 1060 communicating with the axial passage 1076 in the handle portion 1024. The lateral extensions 1072a, 1072b extending from the base portion 1070 provide the handle portion 1024 with a T-handle arrangement to aid the surgeon in grasping and manipulating the instrument 1020. However, it should be understood that other types and configurations of handles are also contemplate for use in association with the instrument 1010, an example of which will be discussed below in association with another embodiment of a surgical instrument 1120.

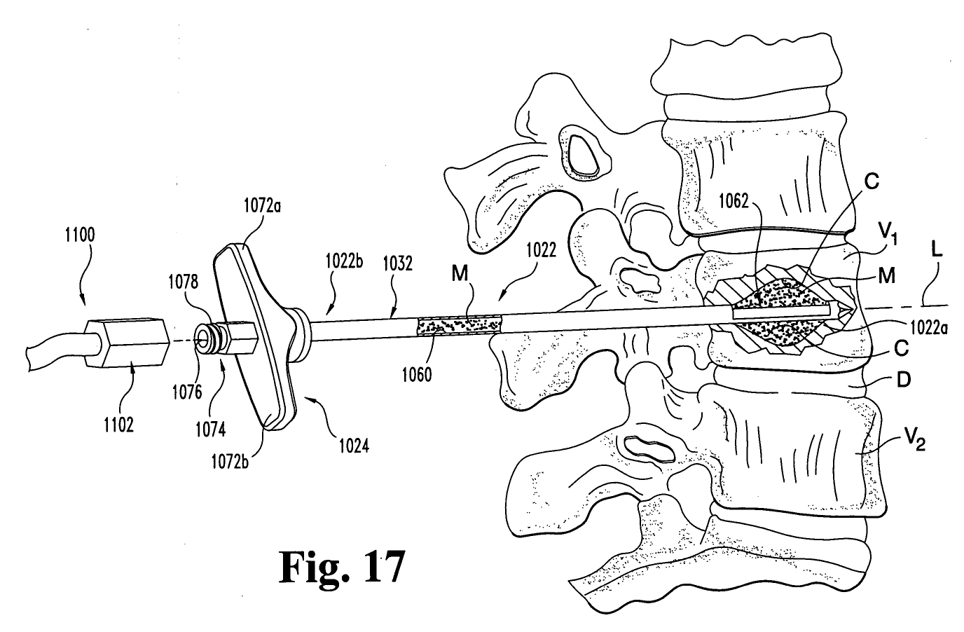

The connector portion 1074 is configured for attachment to a system 1100 (FIG. 17) for delivering material through the instrument 1020 via the axial passage 1076 and the cannula passage 1060 and into a vertebral cavity, the details of which will be discussed below. In the illustrated embodiment, the connector portion 1074 is a lure-type fitting defining external threads 78 adapted for threading engagement with an internally threaded connector element 1102 of the material delivery system 1100 (FIG. 17). However, it should be understood that other types and configurations of connector elements suitable for engagement with a material delivery system are also contemplated as falling within the scope of the invention such as, for example, a bayonet-type fitting, a quick-disconnect fitting, or any other suitable connection arrangement.

As discussed above, the actuator mechanism 1026 serves to selectively transition the deformable strip portion 1038 between the initial and deformed configurations to outwardly expand the deformable strip portion 1038 along the transverse axis T in response to a mechanically induced force provided via selective actuation of the actuator mechanism 1026. In one embodiment of the invention, the actuator mechanism 1026 is generally comprised of an actuator button 1080, a biasing member 1082 and a retaining element 1084. Although a specific embodiment of the actuator mechanism 1026 has been illustrated and described herein, it should be understood that the use of other types and configurations of actuator mechanisms are also contemplated as would occur to one of skill in the art. It should further be understood that in an alternative form of the invention, the inner rod 1030 may be manually engaged by the surgeon, thereby eliminating the need for a separate actuator mechanism 1026.

In one embodiment, the actuator button 1080 includes an engaging portion 1080a, an intermediate portion 1080b, and a spring retaining portion 1080c. The intermediate portion 1080b has an outer cross section that is somewhat smaller than an outer cross section of the engaging portion 1080a so as to define an axially-facing shoulder 1086. Similarly, the spring retaining portion 1080c has an outer cross section that is somewhat smaller than an outer cross section of the intermediate portion 1080b so as to define an axially-facing shoulder 1088. However, it should be understood that other types and configurations of actuator buttons are also contemplated for use in association with the present invention. The actuator rod 1030 extends distally from the actuator button 1080. In one embodiment, the proximal portion 1034 of the actuator rod 1030 is positioned within an axial passage (not shown) extending at least partially through the actuator button 1080, with the actuator rod 1030 attached to the actuator button 1080 via a setscrew 1081 or by any other suitable method of attachment.

In the illustrated embodiment of the invention, the biasing member 1082 is configured as a coil spring. However, it should be understood that other types and configuration of biasing members are also contemplated as would occur to one of ordinary skill in the art. The coil spring 1082 extends about the proximal portion 1034 of the actuator rod 1030. The distal portion of the spring 1082 is positioned about the connector portion 1074 of the handle 1024 and abuts an axially facing surface 1075 of the handle 1024. The proximal portion of the spring 1082 is positioned about the spring retaining portion 1080c of the actuator button 1080 and abuts the axial shoulder 1088. As should be appreciated, the connector portion 1074 and the spring retaining portion 1080c aid in maintaining the spring 1082 in the appropriate position and orientation relative to the handle portion 1024 and the actuator button 1080.

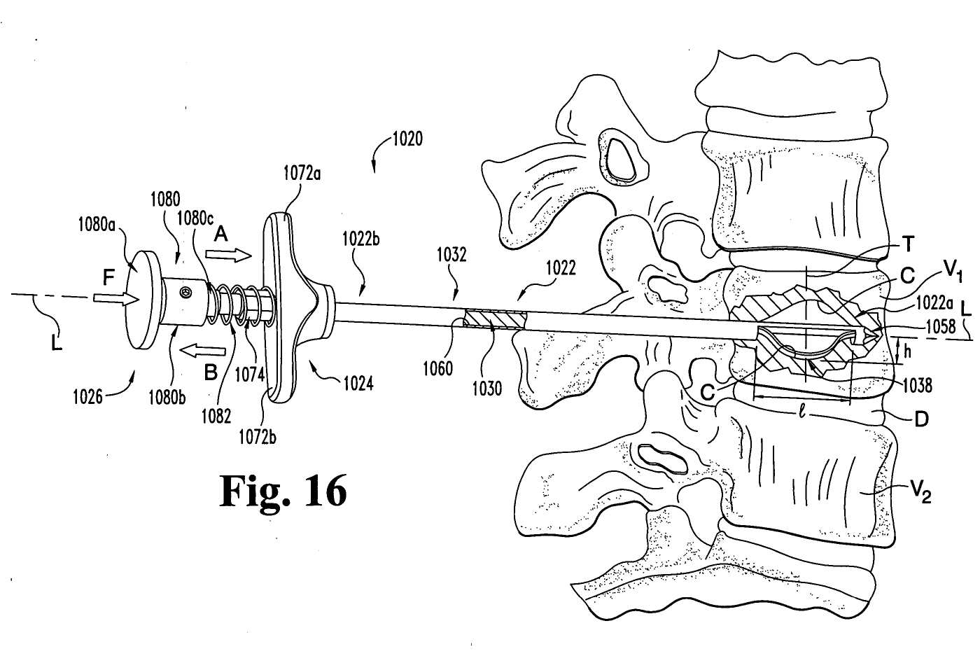

As illustrated in FIG. 16, exertion of an axial force F onto the engaging portion 1080a of the actuator button 1080 correspondingly exerts an axial force onto the actuator rod 1030, which in turn axially displaces the actuator rod 1030 in the direction of arrow A. As should be appreciated, the axial force F may be easily and conveniently provided via grasping of the instrument 1020 with fingers wrapped about the lateral extension 1072a, 1072b of the handle 1024 and with the palm positioned on the engaging portion 1080a of the actuator button 1080. The axial force F is thereby generated by depressing the actuator button 1080 via the surgeon's palm. In this manner, the motion required to generate the axial force F is similar to the motion required to operate a syringe. As should also be appreciated, axial displacement of the actuator button 1080 in the direction of arrow A correspondingly compresses the coil spring 1082 between the handle 1024 and the actuator button 1080, the purpose of which will be discussed below.

Displacement of the actuator rod 1030 in the direction of arrow A results in axial compression of the deformable strip portion 1038 via opposing forces exerted onto the strip portion 1038 by the movable main body portion 1036 and the stationary distal end portion 1040 of the actuator rod 1030. The axial compression force exerted onto the strip portion 1038 in turn causes the strip portion 1038 to outwardly expand or buckle/bow along the transverse axis T. Outward expansion of the strip portion 1038 causes the strip portion 1038 to project through the transverse opening 1062 in the outer sleeve 1032. As should be appreciated, the degree of outward expansion of the strip portion 1038 and the magnitude of the expansion force generated along the transverse axis T can be selectively and accurately controlled by varying the amount of axial force F exerted onto the actuator button 1080. In other words, the amount of axial force F exerted onto the actuator button 1080 by the surgeon is proportional to the degree of outward expansion and the magnitude of the expansion force associated with the strip portion 1038.

Upon removal of the axial force F from the actuator button 1080 via loosening of the surgeon's grip on the engaging portion 1080a and the lateral extensions 1072a, 1072b, the biasing force exerted by the compressed coil spring 1082 onto the actuator button 1080 will correspondingly displace the actuator button 1080 and the actuator rod 1030 in the direction of arrow B. Displacement of the actuator rod 1030 in the direction of arrow B results in removal of the axial compression force on the strip portion 1038, which in turn results in reformation of the strip portion 1038 from the outwardly deformed configuration illustrated in FIG. 16 back toward the initial configuration illustrated in FIG. 13.

Referring once again to FIG. 14, in one embodiment of the invention, the retaining element 1084 is configured to selectively retain the actuator button 1080 and the actuator rod 1030 in a non-actuated position to avoid unintentional deployment or transitioning of the deformable strip portion 1038 toward the outwardly expanded configuration. In the illustrated embodiment, the retaining element 1084 has a clip-like configuration defining a horseshoe shape. However, other shapes and configurations of the retaining elements suitable for selectively maintaining the actuator button 1080 and the actuator rod 1030 in a non-actuated position are also contemplated as falling within the scope of the present invention.

In the illustrated embodiment, the retaining element 1084 has a generally cylindrical sidewall 1090 defining an axial passage 1092 therethrough, and an axial slot 1094 extending the length of the sidewall 1090 so as to define a crosswise opening 1096 communicating with the axial passage 1092. A pair of extension portions or flanges 1098a, 1098b extend from the cylindrical sidewall 1090 in an outwardly tapering manner adjacent the crosswise opening 1096. The crosswise opening 1096 has a minimum opening width that is slightly less than the outer diameter of the intermediate portion 1080b of the actuator button 1080. Additionally, the retaining element 1084 has a length that is substantially equal to the distance between the axially-facing surface 1075 of the handle 1024 and the axially-facing shoulder 1086 of the actuator button 1080.

As should be appreciated, the retaining element 1084 is engagable with the remainder of the instrument 1020 by aligning the crosswise opening 1096 with the proximal portion 1034 of the actuator rod 1030 and transversely displacing the retaining element 1084 to a position between the handle 1024 and the actuator button 1080. The outwardly tapered extension portions 1098a, 1098b of the retaining element 1084 serve to guide the proximal portion 1034 of the actuator rod and the intermediate potion 1080b of the actuator button into the axial passage 1092. As should also be appreciated, since the width of the crosswise opening 1096 is sized slightly less than the outer diameter of the intermediate portion 1080b, the sidewall 1090 of the retaining element 1084 is slightly outwardly deformed to receive the intermediate portion 1080b through the crosswise opening 1096. Once the intermediate portion 1080b is positioned within the axial passage 1092, the sidewall 1090 snaps back into its undeformed condition, thereby selectively engaging the retaining element 1084 to the actuator button 1080. As should further be appreciated, positioning of the retaining element 1084 between the axially-facing surface 1075 of the handle 1024 and the axially-facing shoulder 1086 of the actuator button 1080 selectively retains the actuator button 1080 and the actuator rod 1030 in a non-actuated or non-deployed position.

Having described the components and features associated with the instrument 1020, reference will now be made to a method for using the instrument 1020 in the treatment of a portion of the spine according to one form of the present invention. However, it should be understood that other uses of the instrument 1020 are also contemplated as falling within the scope of the present invention.

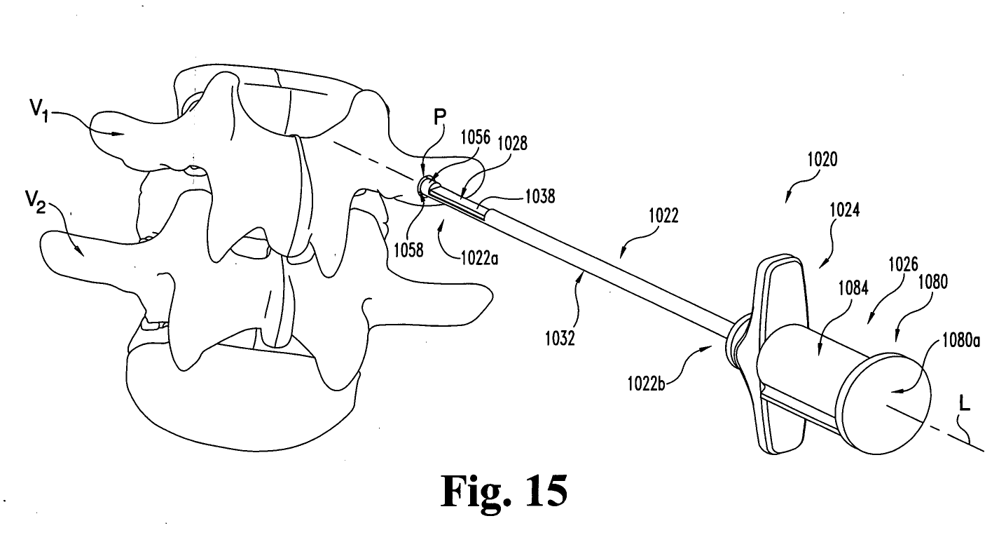

Referring to FIG. 15, shown therein is a posterior view of a portion of a spinal column with the distal portion 1022a of the instrument 1020 being inserted through an access portal P formed through an outer wall of the vertebral body V₁. As discussed above, the retaining element 1084 prevents unintentional deployment or transitioning of the distal portion 1022a of the instrument 1020 toward the outwardly expanded configuration during the initial introduction into the vertebral body V₁. As should also be appreciated, the distal portion 1022a is inserted into the vertebral body V₁ while in the non-expanded initial configuration so as to define a minimal cross-sectional area to minimize the size of the access portal P. When in the non-expanded initial configuration, the distal end portion 1022a has a relatively low profile to facilitate positioning adjacent a vertebral body. As used herein, positioning of the distal end portion 1022a adjacent a vertebral body is meant to include positioning of the distal end portion 1022a in proximity to a vertebral body, within a vertebral body or within a space between adjacent vertebral bodies. In one embodiment of the invention, the initial configuration of the distal end portion 1022a is sized to pass through an access portal having a diameter between about 1 millimeter and about 10 millimeters. In a specific embodiment, the initial configuration of the distal end portion 1022a is sized to pass through an access portal having a diameter of about 5 millimeters. However, other sizes are also contemplated as falling within the scope of the present invention.

In the illustrated embodiment, entry into the vertebral body V₁ is accomplished via a posterior approach and occurs through the pedicle region of the vertebral body V₁. Additionally, entry into the vertebral body V₁ could be either extra-pedicular or trans-pedicular. However, it should be understood that in other embodiments of the invention, entry into the vertebral body V₁ may be accomplished via other surgical approaches such as, for example, an anterior or lateral approach, and could occur through other portions of the vertebral body. Additionally, as indicated above, the instrument 1020 may also be used in interbody applications such as, for example, to distract a portion of the intervertbral space between the adjacent vertebral bodies V₁, V₂.

In one embodiment of the invention, access into the inner region of the vertebral body V₁ is accomplished by drilling a relatively small access portal P through an outer wall of the vertebral body V₁. The undeformed initial configuration of the distal end portion 1022a of the instrument 1020 is sized to pass through the small access portal P to gain access to the inner cancellous region of the vertebral body V₁. In this manner, insertion of the distal end portion 1022a into the vertebral body V₁ is accomplished in a minimally invasive manner. In another embodiment of the invention, access into the inner region of the vertebral body V₁ may be accomplished by driving the pointed tip or trocar portion 1058 of the instrument 1020 into the vertebral body V₁ to form the access portal P via an impaction technique. As should be appreciated, with the retaining element 1084 engaged between the handle 1024 and the actuator button 1080, an impaction force can be exerted onto the engaging portion 1080a of the actuator button 1080 to drive the distal portion 1022a into the vertebral body V₁ while avoiding transitioning of the deformable strip portion 1038 toward the outwardly expanded configuration.

Referring to FIG. 16, once the distal portion 1022a is properly positioned adjacent or within the vertebral body V₁, the retaining element 1084 is removed from the instrument 1020 to allow for selective actuation or deployment of the instrument 1020. Specifically, the distal portion 1022a is transitioned from the initial insertion configuration illustrated in FIG. 15 to the outwardly deformed configuration illustrated in FIG. 16 via exertion of an axial force F onto the engaging portion 1080a of the actuator button 1080 to correspondingly displace the actuator rod 1030 in the direction of arrow A. Axial displacement of the actuator rod 1030 in the direction of arrow A in turn outwardly deforms the distal portion 1022a along the transverse axis T. More specifically, axial compression of the deformable strip portion 1038 causes the strip portion 1038 to outwardly buckle or bow and project through the transverse opening 1062 in the sleeve 1032 so as to define a transverse projection or deformation along the transverse axis T. Since the illustrated embodiment of the instrument 1020 defines a single transverse projection that extends in a single direction, formation of the transverse projection and the resulting preparation of the vertebral body is said to be unidirectional or directionally controlled.

It should be understood, however, that the instrument 1020 may be configured to include multiple transverse projections. In another embodiment, the instrument 1020 may be configured to include a pair of transverse projections extending in generally opposite directions and aligned along a common transverse axis T. In this alternative embodiment, formation of the transverse projections and the resulting preparation of the vertebral body would be described as uniaxial or axially controlled. Although not specifically illustrated herein, it should also be understood that the instrument 1020 may also be configured to include multiple transverse projections positioned at various axial locations along the longitudinal axis L.

As discussed above, outward deformation of the distal portion 1022a along the transverse axis T may be used to compact or compress cancellous bone against the inner cortical wall of the vertebral body to form an intervertebral cavity C therein. Compaction of the cancellous bone also exerts an outward force on the inner surface of the cortical wall adjacent the endplates and/or lateral walls of the vertebral body V₁, thereby making it possible to elevate or push broken and/or compressed bone back to or near its original pre-fracture condition or another desired condition. The deformed distal portion 1022a may also bear directly against the inner surface of the cortical bone to reduce a compression fracture in the vertebral body V₁.

As discussed above, other uses of the instrument 1020 include, for example, distraction of the adjacent vertebral bodies to increase the height of the intervertebral disc space D and/or displacement a spinal implant or other structures used in association with treatment of the spine.

In one embodiment of the invention, the outwardly deformed configuration of the distal portion 1022a has an overall height h along the transverse axis T (as measured from the longitudinal axis L) that falls within a range of about 3 millimeters to about 15 millimeters. In a specific embodiment, the outwardly deformed configuration of the distal portion 1022a has an overall height h of about 7 millimeters. In another specific embodiment of the invention, the instrument 1020 is capable of assuming a deformed configuration having an overall height h that is at least two to three times that of the height of the initial configuration. In another embodiment of the invention, the outwardly deformed configuration of the distal portion 1022a has a length l (as measured along the longitudinal axis L) falling within a range of about 10 millimeters to about 40 millimeters. In a specific embodiment, the outwardly deformed configuration of the distal portion 1022a has an overall length l of about 25 millimeters. Although ranges and specific sizes of the initial and deformed configurations of distal end potion 1022a of the instrument 1020 have been set forth above, it should be understood that such ranges and sizes are exemplary and do not limit the scope of the present invention in any manner whatsoever.

Following formation of the intervertebral cavity C in the vertebral body V₁, the distal end portion 1022a of the instrument 1020 is reformed back toward the initial configuration by displacing the actuator rod 1030 in the direction of arrow B. As discussed above, upon the removal of the axial force F from the actuator button 1080, the biasing force exerted by the compressed coil spring 1082 onto the actuator button 1080 will correspondingly displace the actuator button 1080 and the actuator rod 1030 in the direction of arrow B. Displacement of the actuator rod 1030 in the direction of arrow B results in removal of the axial compression force on the strip portion 1038, which in turn results in reformation of the distal portion 1022a from the outwardly deformed configuration illustrated in FIG. 16 back toward the initial configuration illustrated in FIG. 13. As also discussed above, reformation of the distal portion 1022a back toward the initial configuration may be facilitated by forming at least the strip portion 1038 of a shape-memory material. Once transitioned back to the initial configuration, the distal portion 1022a of the instrument 1020 can be relocated to a different position and/or rotated to a different angular orientation. The instrument 1020 can then be reactivated or redeployed by once again exerting an axial force F onto the actuator button 1080 to outwardly deform the distal portion 1022a along the transverse axis T to enlarge the intervertebral cavity C and/or to form another intervertebral cavity C within the vertebral body V₁.

Following formation of the intervertebral cavity or cavities C, the distal portion 1022a of the instrument 1020 is transitioned back toward the initial configuration illustrated in FIG. 13. In one embodiment of the invention, the instrument 1020 is then removed from the vertebral body V₁. However, as illustrated in FIG. 17, in another embodiment of the invention the inner actuator rod 1030 is removed from the outer sleeve 1032 to define a hollow cannula passage 1060 communicating between the transverse opening 1062 and the axial passages 1076 in the connector portion 1074 of the handle 1024. A material delivery system 1100 may then be attached to the connector portion 1074 to deliver a material M into the axial passage 1076, through the hollow cannula 1060, out the transverse opening 1062 and into the vertebral cavity or cavities C.

Although the illustrated embodiment of the invention depicts the outer sleeve 1032 as defining a single transverse opening 1062 for delivery of the material M into the vertebral cavity C, it should be understood that the sleeve 1032 may define any number of transverse or axial openings for delivery of material M therethrough. It should also be understood that the outer sleeve 1032 may define other types and configurations of delivery openings such as, for example, a plurality of substantially circular opening having a relatively smaller cross section than that of the transverse opening 1062.

As shown in FIG. 17, the material M is delivered into the intervertebral cavity or cavities C to aid in the fixation and structural support of the vertebral body V₁. In one embodiment of the invention, the material M comprises a flowable material that is settable or curable following introduction into the cavity C. Once set to a hardened condition, the material M provides internal structural support to the vertebral body V₁, and more particularly provides structural support to the cortical bone of the vertebral body V₁. In a specific embodiment, the material M comprises a biocompatible filling material such as, for example, a bone cement or various types of synthetic bone material. In another specific embodiment, the material comprises methylmethacrylate cement. However, it should be understood that the material M may comprise other types of materials including, for example, a therapeutic substance to promote healing, a bone growth promoting substance, and/or one or more bone implant support structures.

Although not specifically illustrated in FIGS. 16 and 17, it should be understood that in a further embodiment of the invention, a cannula assembly may be used to provide minimally invasive access to the vertebral bodies V₁, V₂ and/or to the intervertebral disc space D. As should be appreciated, use of a cannula assembly would permit preparation of vertebral tissue via insertion and manipulation of the instrument 1020 and other instrumentation or device through a single working channel.

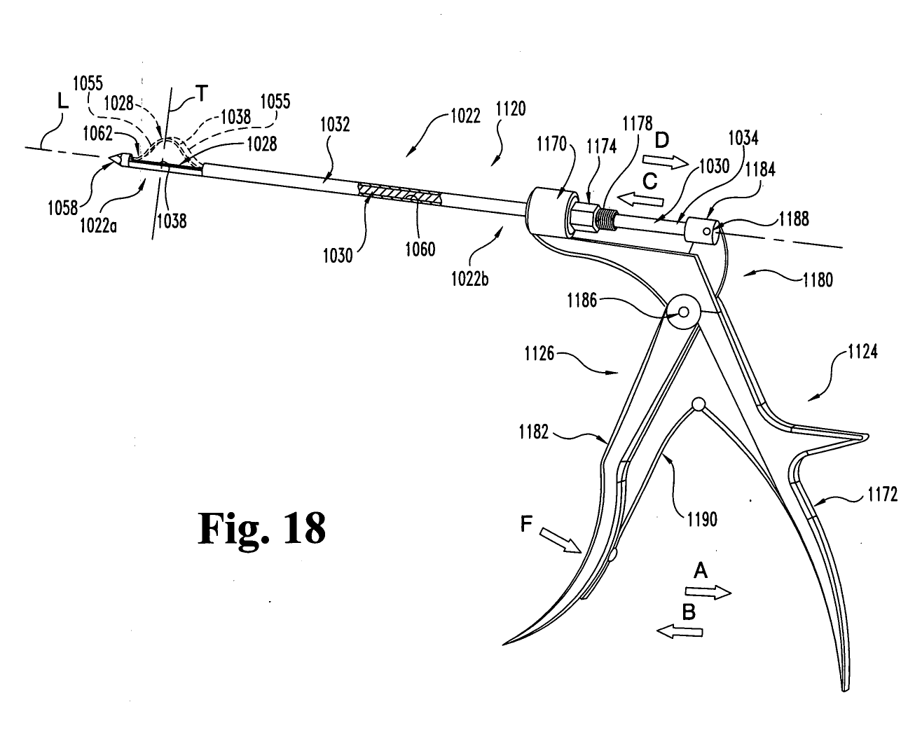

Referring to FIG. 18, shown therein is an instrument 1120 for treatment of the spine according to another form of the present invention. The instrument 1120 is used in association with applications such as those discussed above with regard to the instrument 1020, and is particularly useful for placement adjacent a spinal structure to selectively displace at least a portion of the spinal structure. In many respects, the illustrated embodiment of the instrument 1120 is structurally and functionally similar to the instrument 1020 illustrated and described above. Accordingly, like elements and features are indicated and referred to using the same reference numerals.

Similar to the instrument 1020, the instrument 1120 is generally comprised of an elongate member 1022, a handle portion 1124, an actuator mechanism 1126, and a deformable portion 1028 that is selectively transitionable between an initial configuration (shown in solid lines) and a deformed configuration (shown in phantom lines). The elongate member 1022 extends generally along a longitudinal axis L and has a distal portion 1022a and a proximal portion 1022b. The handle portion 1124 aids in the manipulation and handling of the instrument 1120 and also includes a mechanism for connecting to a material delivery system, the detail of which will be discussed below. The actuator mechanism 1126 serves to transition the deformable portion 1028 between the initial and deformed configurations. The deformable portion 1028 is positioned adjacent the distal portion 1022a of the elongate member 1022 and outwardly expands along the transverse axis T in response to a mechanically induced force that is provided via selective actuation of the actuator mechanism 1126.

In the illustrated embodiment, the handle portion 1124 and the actuator mechanism 1126 have a kerrison-type configuration. Specifically, the handle portion 1124 is generally comprised of a base portion 1170, a grip portion 1172, and a connector portion 1174. The handle portion 1124 includes an axial passage (not shown) extending through the base portion 1170 and the connector portion 1174, with the outer sleeve 1032 extending distally from the base portion 1170. The cannula passage of the outer sleeve 1032 communicates with the axial passage extending through the base portion 1170 and the connector portion 1174. The grip portion 1172 aids the surgeon in grasping and manipulating the instrument 1120. The connector portion 1174 is configured for attachment to a system 1100 (FIG. 17) for delivering a material M through the instrument 1120 and into one or more vertebral cavities C. In the illustrated embodiment, the connector portion 1174 comprises a lure-type fitting defining external threads 1178 adapted for threading engagement with an internally threaded connector element 1102 of the material delivery system 1100 (FIG. 17). However, it should be understood that other types and configurations of connector elements suitable for engagement with a material delivery system are also contemplated as falling within the scope of the invention such as, for example, a bayonet-type fitting, a quick-disconnect fitting, or any other suitable connection arrangement.

As discussed above, the actuator mechanism 1126 serves to selectively transition the deformable strip portion 1038 between the initial and deformed configurations to outwardly expand the deformable strip portion 1038 along the transverse axis T in response to a mechanically induced force provided via selective actuation of the actuator mechanism 1126. In one embodiment of the invention, the actuator mechanism 1126 is generally comprised of an actuator or trigger portion 1180 and a biasing member 1190. Although not specifically illustrated in FIG. 18, the actuator mechanism 1126 may also include a retaining element configured to selectively retain the trigger portion 1180 and the actuator rod 1030 in a non-actuated position to avoid unintentional deployment or transitioning of the deformable strip portion 1038 toward the outwardly expanded configuration. The trigger portion 1180 generally includes a grip portion 1182 and a coupler portion 1184. The grip portion 1182 is pivotally attached to the grip portion 1172 of the handle 1124 via a pivot pin 1186 to allow for relative pivotal movement therebetween in the direction of arrows A and B. The grip portion 1182 is pivotally attached to the coupler portion 1184 via a pivot pin 1188 to provide pivotal engagement between the proximal end 1034 of the actuator rod 1030 and the grip portion 1182.

In the illustrated embodiment, the biasing member 1190 is configured as a U-shaped strip-like spring element. However, it should be understood that other types and configuration of biasing members are also contemplated as would occur to one of ordinary skill in the art including, for example, a coil spring. The spring 1190 is engaged between the grip portions 1172, 1182 and serves to bias the grip portions 1172, 1182 apart to maintain the instrument 1120 in a non-actuated or non-deployed configuration. However, exertion of a force F onto the grip portion 1182 causes the grip portion 1182 to pivot in the direction of arrow A, which in turn exerts an axial force onto the proximal portion 1034 of the actuator rod 1030 to displace the actuator rod 1030 in the direction of arrow C. Displacement of the actuator rod 1030 in the direction of arrow C results in axial compression of the deformable strip portion 1038, which in turn causes the strip portion 1038 to outwardly expand or buckle/bow along the transverse axis T. As should be appreciated, the degree of outward expansion of the strip portion 1038 and the magnitude of the expansion force generated along the transverse axis T can be selectively and accurately controlled by varying the amount of force F exerted onto the grip portion 1182. In other words, the amount of force F exerted onto the grip portion 1182 by the surgeon is proportional to the degree of outward expansion and the magnitude of the expansion force associated with the deformed strip portion 1038.

In the illustrated embodiment, the force F exerted onto the grip portion 1182 is provided via grasping of the instrument 1120 with fingers wrapped about the grip portion 1182 and with the palm and/or thumb positioned on the grip portion 1172. The axial force F is thereby generated by squeezing the grip portion 1182 toward the grip portion 1172. As should be appreciated, pivotal movement of the grip portion 1182 in the direction of arrow A correspondingly compresses the spring element 1190 between the grip portions 1172, 1182. As should also be appreciated, upon removal of the force F via loosening of the surgeon's grip on the grip portion 1182, the biasing force exerted by the compressed spring 1190 will correspondingly displace the grip portion 1182 in the direction of arrow B, which in turn displaces the actuator rod 1030 in the direction of arrow D. Displacement of the actuator rod 1030 in the direction of arrow D results in removal of the axial compression force on the strip portion 1038, which in turn results in reformation of the strip portion 1038 from the outwardly deformed configuration (as shown in phantom lines) back toward the initial configuration (as shown in sold lines).

In some embodiments of the invention, the deformable strip 1038 may be designed to provide a cutting edge 1055 that is exposed to cut tissue when the deformable strip 1038 is extended. The cutting edge 1055 may be a thin portion of the deformable strip 1038, or in some embodiments may be sharpened to an edge that is significantly thinner than the thickness of the deformable strip 1038 to provide a sharper cutting edge. The cutting edge may be tempered, serrated, or otherwise treated or configured to enhance the ability of the projections to cut through tissue, as is known in the art of tissue cutting devices.

Similar to the instrument 1020 illustrated and described above, the instrument 1120 is configured to allow for removal of the inner actuator rod 1030 from the outer sleeve 1032 to provide an axial passageway 1060 for the delivery of a material M into the intervertebral cavity C formed within the vertebral body V₁. Specifically, following transitioning of the distal portion 1022a of the instrument 1120 back to the initial configuration, the actuator rod 1030 is removed from the outer sleeve 1032 to define a hollow cannula 1060 communicating between the transverse slotted opening 1062 and the connector portion 1074. A material delivery system 1100 (FIG. 17) may then be attached to the connector portion 1074 to deliver a material M through the hollow cannula 1060, out the transverse opening 1062 and into the vertebral cavity C.

As should now be appreciated, in the illustrated embodiments of the invention, the instruments 1020, 1120 are capable of performing multiple functions associated with treatment of the spine. For example, the trocar 1058 facilitates entry into and through vertebral tissue. Additionally, the deformable distal portion 1022a, and more specifically the deformable strip portion 1038, serves to reduce a vertebral fracture and/or to form one or more intervertebral cavities C with the vertebral body V₁. Further, upon the selective removal of the inner actuator rod 1030, the outer sleeve 1032 provides a hollow cannula 1060 for delivering a material M into the intervertebral cavity C. As should be appreciated, the use of a single instrument to perform multiple functions associated with a spinal treatment procedure tends to simplify the surgical procedure, lessen the time required to perform the procedure, and/or reduce the costs and expenses compared to providing multiple surgical instruments to perform similar functions. Additionally, if the instrument 1020, 1120 is designed as a single use instrument, the cost associated with sterilizing the instrument 1020, 1120 for reuse are eliminated.

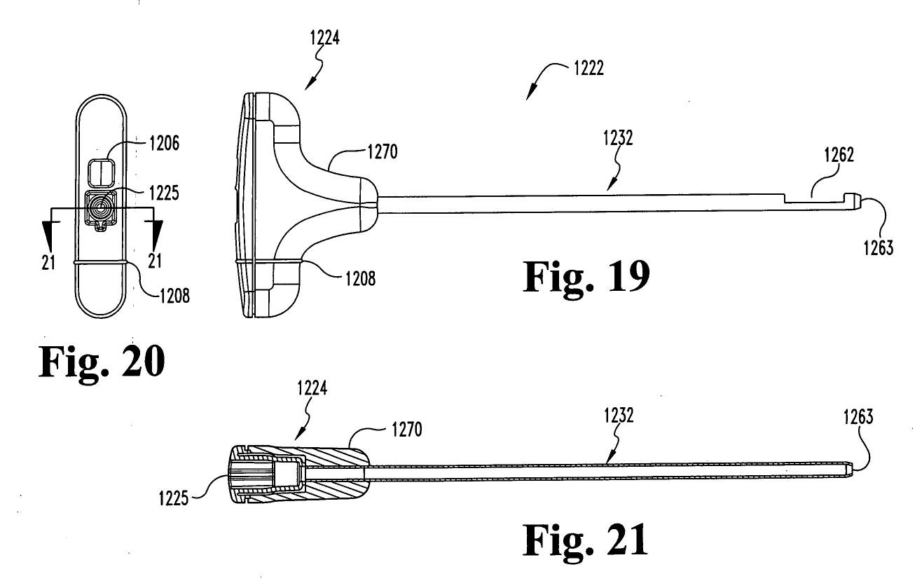

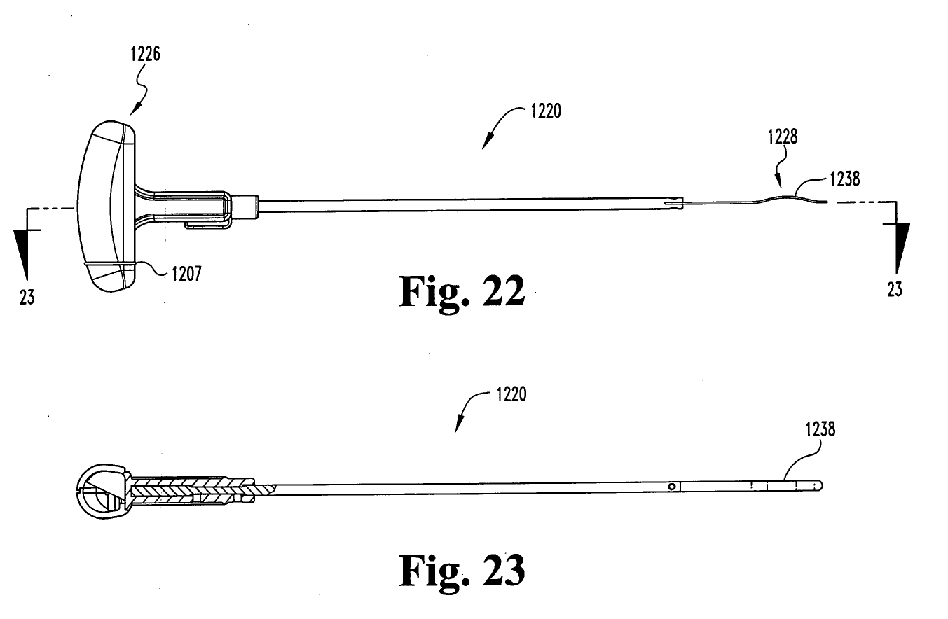

Another embodiment of the invention is illustrated in FIGS. 19-27. Instrument 1220 is configured for treatment of the spine according to another form of the present invention. The instrument 1220 is used in association with applications such as those discussed above with regard to the instrument 1020, and is particularly useful for placement adjacent a spinal structure to selectively prepare or displace at least a portion of the spinal structure.

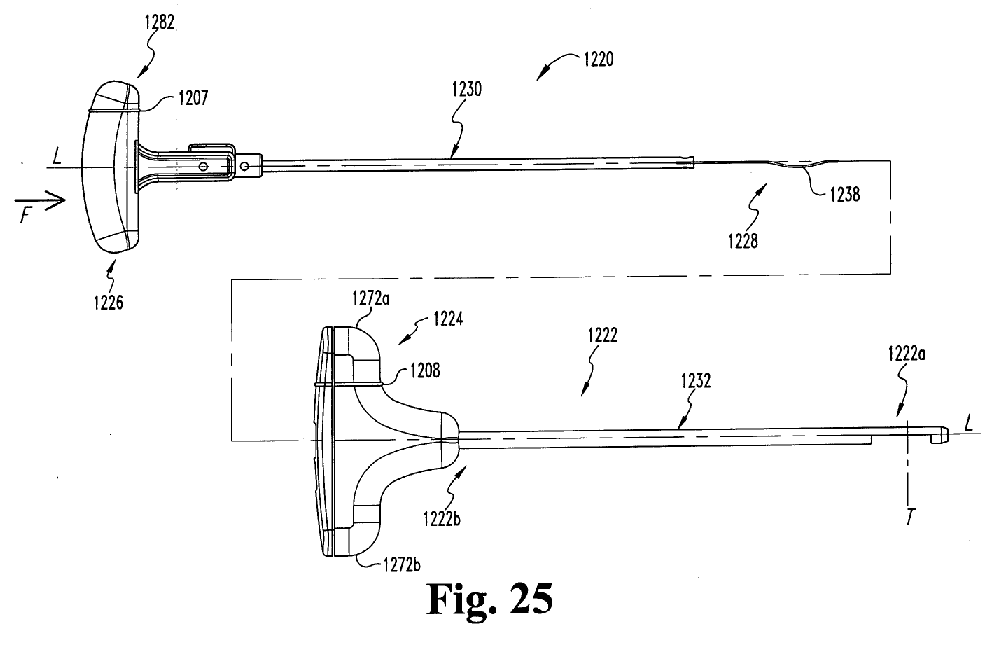

Similar to the instrument 1020, the instrument 1220 (FIG. 25) is generally comprised of an elongate member 1222, a handle portion 1224, an actuator mechanism 1226, and a deformable portion 1228 that is selectively transitionable between an initial configuration and a deformed configuration. The elongate member 1222 extends generally along a longitudinal axis L and has a distal portion 1222a and a proximal portion 1222b. The handle portion 1224 aids in the manipulation and handling of the instrument 1220. The actuator mechanism 1226 serves to transition the deformable portion 1228 between the initial and deformed configurations. The deformable portion 1228 is positioned adjacent the distal portion 1222a of the elongate member 1222 and outwardly expands along the transverse axis T in response to a mechanically induced force that is provided via selective actuation of the actuator mechanism 1226.

Referring now to FIGS. 19-21, the handle portion 1224 includes an axial passage 1225 extending through the base portion 1270, with the outer sleeve 1232 extending distally from the base portion 1270. The cannula passage of the outer sleeve 1232 communicates with the axial passage extending through the base portion 1270. The grip portion 1272 aids the surgeon in grasping and manipulating the instrument 1220.

As discussed above, the actuator mechanism 1226 serves to selectively transition a deformable strip portion 1238 of the deformable portion 1228 between the initial and deformed configurations to outwardly expand the strip 1238 along the transverse axis T in response to a mechanically induced force provided via selective actuation of the actuator mechanism 1226.

In the embodiment illustrated in FIG. 25, a force F exerted onto the grip portion 1282 is provided via grasping of the instrument 1220 with fingers wrapped about the grip extensions 1272a, 1272b and with the palm positioned on the grip portion 1282. The axial force F is generated by squeezing the grip portion 1282 toward the grip extensions 1272a, 1272b. The axial force F thereby transfers force through the inner actuator rod 1230 and transitions the strip portion 1238 to a deformed configuration. As should also be appreciated, upon removal of the force F via loosening of the surgeon's grip on the grip portion 1282, the biasing force exerted by the strip portion 1238 will correspondingly displace the grip portion 1282 proximally. In this manner, the strip portion 1238 also acts as a spring.

In some embodiments of the invention, the deformable strip 1238 may be designed to provide a cutting edge 1255 that is exposed to cut tissue when the deformable strip 1238 is extended. The cutting edge 1255 may be a thin portion of the deformable strip 1238, or in some embodiments may be sharpened to an edge that is significantly thinner than the thickness of the deformable strip 1238 to provide a sharper cutting edge. The cutting edge may be tempered, serrated, or otherwise treated or configured to enhance the ability of the projections to cut through tissue, as is known in the art of tissue cutting devices.

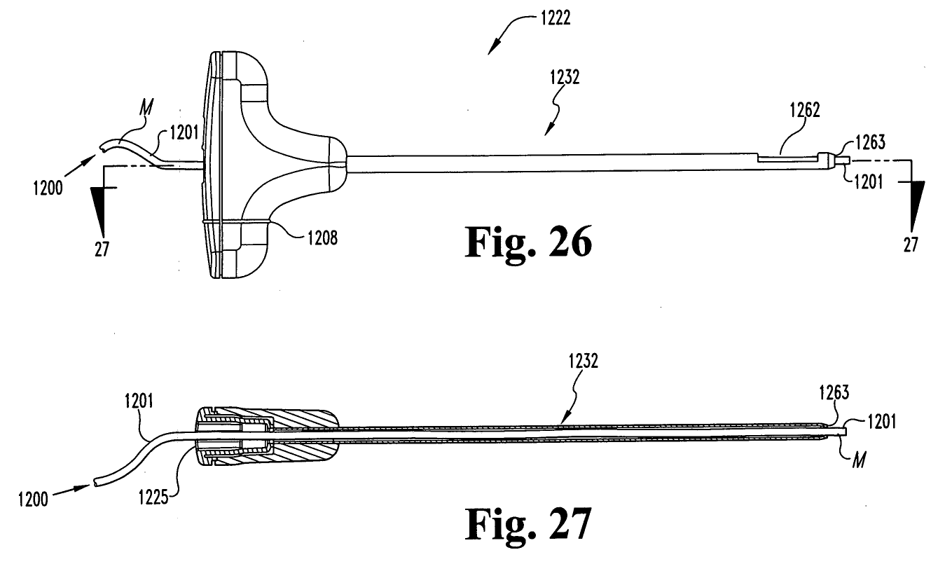

Similar to the instrument 1020 illustrated and described above, the instrument 1220 is configured to allow for removal of the inner actuator rod 1030 from the outer sleeve 1232 to provide an axial passage 1225 (FIGS. 20-21) for the delivery of a material M into the spinal structure. Specifically, following transitioning of the distal portion 1222a (including strip portion 1238) of the instrument 1220 back to the initial configuration, the actuator rod 1230 is removed from the outer sleeve 1232 to define an axial passage 1225 communicating between a transverse slotted opening 1262 and the proximal end of elongated member 1222 (FIGS. 26 and 27). A material delivery system 1200, such as an injector, may then be used to deliver a material M through the axial passage 1225 and out of the transverse slotted opening 1262 and/or the distal opening 1263. In the illustrated embodiment, the material M is being delivered through a delivery tube 1201 that is connected to the material delivery system 1200.

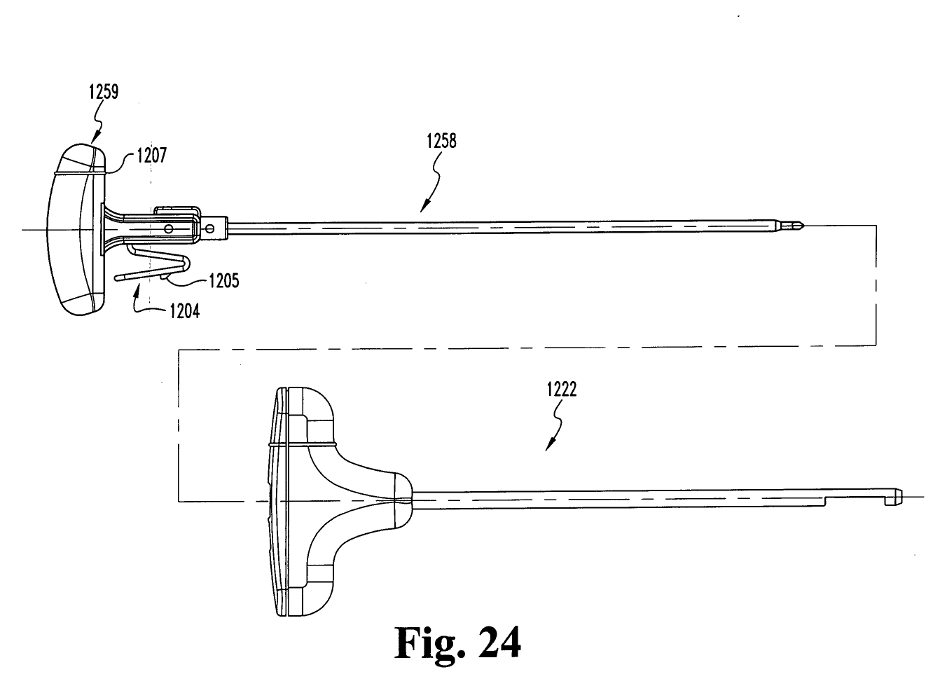

As should now be appreciated, in the illustrated embodiments of the invention, the instrument 1220 is capable of performing multiple functions associated with treatment of the spine. For example, FIG. 24 shows a stylet 1258 for facilitating entry into and through vertebral tissue. The stylet 1258 is insertable in the elongated member 1222 and the combined devices are used to locate an access point and to enter vertebral tissue. The stylet 1258 and the elongated member 1222 connect together via the locking mechanism 1204. Locking mechanism 1204 includes a catch 1205 that locks into hole 1206 (FIG. 20) when stylet 1258 is inserted into elongated member 1222. When proper positioning is achieved, the stylet 1258 can be removed from the elongated member 1222 and the elongated member 1222 can remain in place to provide a cannula into the vertebral tissue.

Each of the handle 1259 of the stylet 1258 and the grip portion 1282 include alignment markings 1207 that indicate to a user the proper alignment of the devices in the elongated member 1222. The elongated member includes a corresponding alignment base 1208 which, when aligned with the alignment markings 1207 of the appropriate device, ensures correct assembly of the instrument.

Additionally, the deformable distal portion 1222a, and more specifically the deformable strip portion 1238, may be used to loosen or cut vertebral tissue to reduce a vertebral fracture and/or to form one or more intervertebral cavities in a vertebral body. As should be appreciated, the use of a single instrument to perform multiple functions associated with a spinal treatment procedure tends to simplify the surgical procedure, lessen the time required to perform the procedure, and/or reduce the costs and expenses compared to providing multiple surgical instruments to perform similar functions. Additionally, if the instrument 1220 is designed as a single use instrument, the costs associated with sterilizing the instrument 1220 for reuse are eliminated.

Embodiments of the invention include various methods of use of the disclosed devices. The various methods may be operable with one or more of the instruments of the disclosed embodiments of the invention.

In one method embodiment, treatment of the spine is accomplished by use of an instrument defining a cannula passage extending along a longitudinal axis and including a deformable distal portion having an insertion configuration and a deformed configuration. For example, the instruments of at least FIGS. 13, 18, and 25 would provide such features.

In some embodiments, the spine is treated by at least the acts of providing an instrument defining a cannula passage extending along a longitudinal axis and including a deformable distal portion having an insertion configuration and a deformed configuration. Further, the distal portion of the instrument is positioned within a spinal structure while in the insertion configuration, and the instrument is activated to loosen tissue within the spinal structure. A material may then be delivered through the cannula passage and into the spinal structure.

In accomplishing certain method embodiments, the distal portion of an instrument is positioned within a spinal structure while in an unexpanded or “insertion configuration.” Actuation of an instrument then transitions the distal portion of the instrument toward an expanded or deformed configuration. Simultaneously with the expansion of the instrument, the instrument is rotated about the longitudinal axis. Rotation of the expanding instrument contacts tissue in the path of the expanded components and loosens tissue within the spinal structure. In some embodiments, loosened material is removed by either suction or mechanical engagement with the loosened material. Equipment to generate a suction force is available in many operating rooms through a tubing suction system. Alternatively, suction may be generated with a syringe or by any other effective means.

In some method embodiments, the interior of a vertebral body or other structure is irrigated with a fluid to manipulate the contents of the structure. For example, a solution may be used to clean the inside of a vertebral body. The solution may be injected through the cannula to suspend loosened material within the vertebral body, and then suctioned back through the cannula. Alternatively, the suctioning may be provided simultaneously with the injection of the fluid. By way of example, the fluid may be provided through a cannula inserted through one pedicle while a suction tube inserted through the contralateral pedicle removes fluid.

One or more fluids may be injected to created desirable therapeutic results. A saline solution could be used to clean the inside of a vertebral body by circulation though the vertebral body. Subsequently, another fluid such as, for example, air or inert gas may be injected into or circulated through the vertebral body. The additional fluid may be, without limitation, effective to facilitate hemostasis, to better prepare the vertebral body to accept a therapeutic agent, or to dry the tissue within the vertebral body. One or more of the fluids used may contain biologically and/or chemically active substances useful to create a desired clinical result.

With a loosened or evacuated volume within the spinal structure created, material may be delivered through the cannula passage and into the spinal structure. Several specific and adequate bone fillers are detailed in the disclosure, and additionally, the delivered material may be any material that creates a positive therapeutic result for a patient.

For the purposes of the following description, transitioning the deformable distal portion to a deformed configuration will be referred to as expanding the instrument, and transitioning the distal portion substantially to the insertion configuration will be referred to as returning to the insertion configuration. Note that the instrument need not be returned to precisely the same degree of expansion as when inserted to be returned to the insertion configuration as used herein. Loosening of the tissue within the spinal structure may be accomplished by expanding the instrument in a first location, returning the instrument to the insertion configuration, and rotating the instrument about the longitudinal axis to a second location. In the second location, the instrument is again expanded. While expanded, the instrument is rotated about the longitudinal axis at least to the first location. This procedure may be repeated one or more times to create a volume of loosened tissue.

Loosening of the tissue within the spinal structure may also be accomplished by expanding the instrument and rotating the instrument one or more revolutions as needed to loosen the tissue.

Loosening of the tissue within the spinal structure may additionally be accomplished by expanding the instrument and rotating the instrument any degree of rotation about the longitudinal axis. The instrument may then be expanded to a second deformed configuration that has a greater transverse deformation or is larger. In the position of larger expansion, the instrument is again rotated to some degree about the longitudinal axis. Such a technique may be beneficial where the method is accomplished in a patient with relatively strong bony structure that produces greater resistance to rotation.

Loosening of the tissue within the spinal structure may additionally be accomplished by expanding and rotating the instrument about the longitudinal axis, and then changing the degree of the expansion to a second deformed configuration. The second deformed configuration may be greater or lesser than the first expansion. In the second configuration, the instrument is rotated about the longitudinal axis. This procedure may be useful to avoid certain portions with a body being loosened, when the instrument is not symmetrically placed in the bony structure, and at other times.

In another embodiment, treatment of the spine is accomplished by use of an instrument defining a cannula passage extending along a longitudinal axis and including a deformable distal portion having an insertion configuration and a deformed configuration. The distal portion of the instrument is positioned within a spinal structure while in the insertion configuration. The instrument is expanded, returned to its insertion configuration, and then rotated about the longitudinal axis. In the new position, the instrument is expanded and released again. This expansion, release, and rotation is repeated until the deformable distal portion has been deployed to contact more than half of the radial surface of the interior of the spinal structure. When the material inside the spinal structure has been manipulated as desired, material is delivered through the cannula passage and into the spinal structure. This embodiment is useful, among other functions, to move tissue away from the center of the spinal structure.

Another method of the invention is designed to enhance the placement of filler material within a spinal structure in an effective manner. The method includes providing an instrument with a cannula passage extending along a longitudinal axis. The distal portion of the instrument is positioned within a spinal structure. A first portion of filler material is delivered through a tube extended through the cannula to a distal end of the accessible portion of the spinal structure. In some embodiments, this delivery may be monitored with fluoroscopy, endoscopy, or by any effective means. The tube is withdrawn proximally relative to the cannula when deemed appropriate by the surgeon. In some cases, the withdrawal of the tube will allow the filler material to be more directly placed near its final position and therefore prevent the building of pressure in the spinal structure during a procedure. In a withdrawn position, a second portion of filler material is delivered through the tube and into the spinal structure.

Another embodiment of the invention is an actuator for manipulating tissue. The actuator includes at least a first member extending in the direction of a longitudinal axis and a second member extending in the direction of the longitudinal axis. The first member and the second member are movable relative to one another along some portion in the direction of the longitudinal axis. Embodiments of the actuator have a deformable distal portion of the actuator with a strip with a greatest dimension substantially in the direction of the longitudinal axis. The strip buckles to create a transverse projection when the first member and the second member are moved relative to one another in the direction of some portion of the longitudinal axis. The strip also has a cutting edge that is exposed to the tissue when the first member and the second member are moved relative to one another along some portion in the direction of the longitudinal axis.

While the invention has been illustrated and described in detail in the drawings and foregoing description, the same is to be considered as illustrative and not restrictive in character.

What is claimed is:

1. A kit for treatment of the spine, comprising:
a cannula for maintaining a passageway to a portion of the spine to be treated;
a surgical instrument for providing surgical access to the spine, the instrument being operable through the cannula;
a bone filler injector; and
a tube that provides a conduit between the bone filler injector and the cannula;
wherein the tube is extendable through the cannula to a position adjacent to the portion of the spine to be treated.

2. The kit of claim 1 wherein at least a portion of a distal end of the cannula has an opening through which the instrument operates.

3. The kit of claim 2 wherein the opening is in a distal most portion of the distal end of the cannula.

4. The kit of claim 1 wherein the surgical instrument is a stylet.

5. The kit of claim 1 wherein the surgical instrument is a spring.

6. The kit of claim 1 wherein the bone filler injector provides bone filler at a controlled pressure and volume.

7. The kit of claim 1 wherein the tube has an outside diameter of greater than 0.5 mm less than the inside diameter of the cannula.

8. The kit of claim 1 wherein the cannula has an opening through which a bone filler material is injectable into the spine.

9. The kit of claim 8 wherein the opening is in a distal most portion of the distal end of the cannula.

10. The kit of claim 1 wherein the cannula has an opening through which the tube may be extended.

11. The kit of claim 10 wherein the opening is in a distal most portion of the distal end of the cannula.

12. The kit of claim 1 wherein the tube is a flexible tube.

13. A method of performing a biopsy, comprising:
providing a medical instrument comprising:
a cannula member extending along a longitudinal axis and including a distal portion, said cannula member defining an axial passage and a transverse opening positioned adjacent said distal portion and communicating with said axial passage; and
an actuator member removably positioned within said axial passage of said cannula member and including a deformable portion positioned adjacent said transverse opening, said deformable portion being transitionable between an initial configuration for placement within a spinal structure and a deformed configuration defining a transverse projection extending through said transverse opening in said cannula member; and
selectively removing tissue on which a biopsy is to be accomplished from said cannula member.

14. A method for treatment of the spinet comprising:
providing an instrument defining a cannula passage extending along a longitudinal axis and including a deformable distal portion having an insertion configuration and a deformed configuration;
positioning the distal portion of the instrument within a spinal structure while in the insertion configuration;
transitioning the distal portion of the instrument toward the deformed configuration while simultaneously rotating the instrument about the longitudinal axis to form a volume of loosened tissue within the spinal structure; and
delivering a material through the cannula passage and into the spinal structure.

15. The method of claim 14 further comprising removing a portion of the loosened tissue from the spinal structure prior to delivering a material into the spinal structure.

16. The method of claim 15 wherein removing a portion of the loosened tissue includes suctioning loosened tissue.

17. The method of claim 15 wherein removing a portion of the loosened tissue includes mechanically removing loosened tissue.

18. A method for treatment of the spine, comprising:
providing an instrument defining a cannula passage extending along a longitudinal axis and including a deformable distal portion having an insertion configuration and a deformed configuration;
positioning the distal portion of the instrument within a spinal structure while in the insertion configuration;
activating the instrument to loosen tissue within the spinal structure;
removing a portion of the loosened tissue from the spinal structure; and
delivering a material through the cannula passage and into the spinal structure.

19. The method of claim 18 further comprising injecting a fluid into the spinal structure to prepare the structure to receive a delivered material.

20. The method of claim 19 wherein injecting a fluid includes injecting a liquid.

21. The method of claim 19 wherein injecting a fluid includes injecting a gas.

22. A method for treatment of the spine, comprising:
providing an instrument defining a cannula passage extending along a longitudinal axis;
positioning the distal portion of the instrument within a spinal structure while in the insertion configuration;
delivering a first portion of filler material through a tube extended through the cannula to a distal end of an accessible portion of the spinal structure;
withdrawing the tube proximally relative to the cannula; and
delivering a second portion of filler material through the tube and into the spinal structure.
